(19) United States
(12) Patent Application Publication
(10) Pub. No.: US 2007/0073047 A1
(43) Pub. Date: Mar. 29, 2007

(54) Genes overexpressed by ovarian cancer and their use in developing novel therapeutics

(75) Inventors: Hariharan Kandasamy, San Diego, CA (US); Mark Daniels, San Diego, CA (US); Karen McLachlan, Del Mar, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 10/530,951

(22) PCT Filed: Jun. 10, 2003
(86) PCT No.: PCT/US03/18253

§ 371 Date: Aug. 18, 2006

Related U.S. Application Data
(60) Provisional application No. 60/386,748, filed on Jun. 10, 2002.
(60) Provisional application No. 60/396,141, filed on Jul. 17, 2002.
(60) Provisional application No. 60/405,319, filed on Aug. 23, 2002.
(60) Provisional application No. 60/428,274, filed on Nov. 22, 2002.

(51) Int. Cl. A61K 48/00; C07K 14/82; C07K 16/30; C12Q 1/68; G01N 33/574; C07H 21/04; C12P 21/06; A61K 39/395

(52) U.S. Cl. 536/23.5; 514/44; 435/6; 435/69.1; 435/320.1; 435/325; 530/350; 530/388.8; 424/155.1; 435/7.23

(57) ABSTRACT

Nucleic acid sequences encoding genes that are overexpressed by human ovarian cancers are provided. These genes and the corresponding antigens are useful diagnostic and therapeutic targets. The invention provides cancer therapies that target these antigens, especially using monoclonal antibodies that target the Anat-2 antigen.

### CROSS-REFERENCE TO RELATED APPLICATIONS

This application claims priority to U.S. Provisional Ser. No. 60/386,748 filed Jun. 10, 2002, U.S. Provisional Ser. No. 60/396,141 filed on Jul. 17, 2002, U.S. Provisional Ser. No. 60/405,319 filed on Aug. 23, 2002 and U.S. Provisional Ser. No. 60/428,274 filed on Nov. 22, 2002, each of which is incorporated by reference in its entirety. Related applications U.S. Ser. No. 10/326,924, filed Dec. 23, 2002, and U.S. Provisional Ser. No. 60/341,860, filed Dec. 21, 2001, now lapsed, are also incorporated by reference in their entirety.

### FIELD OF THE INVENTION

The present invention relates to the identification of genes that are upregulated in ovarian cancer. These genes or the corresponding proteins are to be targeted for the treatment, prevention and/or diagnosis of cancers wherein these genes are upregulated, particularly ovarian cancer.

### BACKGROUND OF THE INVENTION

Ovarian cancer is a disorder that affects thousands of women annually. Unfortunately, it is a cancer that is usually not detected until the disease has progressed to a fairly advanced stage.

Consequently, a large percentage of women diagnosed with the disease do not survive.

Currently, there do not exist may effective therapies for ovarian cancer. Generally, treatment of ovarian cancer comprises surgical removal of the ovaries and any other tissues to which the cancer may have spread, followed by chemotherapy or radiation or a combination thereof. For example, the use of Taxol and certain growth factors or hormones, e.g., progestin and EGF in treatment of ovarian cancer have been reported.

In the past ten to fifteen years, various gene targets have been identified, the presence of which correlates to the presence of particular types of ovarian cancers.

For example, it has been reported that specific BRCA2 gene alleles correlate to persons having a predisposition to develop breast and ovarian cancer. (See U.S. Pat. No. 6,045,997, issued Apr. 4, 2000, to Futreol et al. and assigned to Duke University and Cancer Research Campain Technology Limited.) Also, it has been reported that the presence of specific erbB-2 genes, and ligands thereto correlate to a predisposition for developing breast and ovarian cancer, and that these genes and ligands are useful targets for treatment and diagnosis. (See U.S. Pat. No. 6,040,290, issued Mar. 27, 2000, to Lippman et al., assigned to Georgetown University, which teaches ligand growth gp30 that binds to erbB-2 receptor protein; U.S. Pat. No. 6,037,134, issued Mar. 17, 2000, to Margolis and U.S. Pat. No. 6,001,583 issued Dec. 14, 1999, assigned to New York University, Medical Center, which teach HER2/GRB-7 complexes, the presence of which correlates to certain breast and ovarian cancers; and U.S. Pat. Nos. 5,772,997, 5,770,195 issued to Hudziak and assigned to Genentech, issued respectively on Jun. 30, 1998 and Jun. 23, 1998, as well as U.S. Pat. Nos. 5,725,856 and 5,729,954, issued respectively on Mar. 10, 1998 and Feb. 24, 1998, and assigned to Genentech, which teach monoclonal antibodies to HER2 receptor.

Further, the use of antisense oligonucleotides to treat cancers including breast and ovarian carcinomas has been reported, e.g., U.S. Pat. No. 6,007,997, issued Dec. 28, 1999, to Sivaraman et al. and assigned to the Research Foundation of SUNY, which discloses the use of antibodies oligos complementary to ERR-1 or ERR-2 to treat ovarian and breast cancer. Also, U.S. Pat. No. 5,968,748 to Bennett et al., assigned to ISIS Pharmaceutical and Pennsylvania State Research Foundation, discloses the use of HER2 anti-sense oligos to treat breast and ovarian cancers.

Still further, it has been reported that TAT1 (tumor associated trypsin inhibitor) is a marker of ovarian cancer (Medl et al., Br. J. Cancer 71: 1051-1054 (1995)). Also, the use of EGFR as a target for advanced ovarian cancer has been reported (Scambia et al., J. Clin Oncol, 10: 529-535 (1992).

Moreover, BRCA-1 protein kinase has been reported to be a useful diagnostic and treatment target for ovarian cancer. (See U.S. Pat. No. 5,972,675 issued Oct. 26, 1999 to Backmann et al., assigned to Eli Lilly and Company; U.S. Pat. No. 5,891,857 issued Apr. 6, 1999 to Holt et al., and jointly assigned to Vanderbilt University and the University of Washington.) Additionally, another useful target for treating cancers affecting the female genital tract is reported in U.S. Pat. No. 5,814,315 issued Sep. 29, 1999 to Hing, et al. and assigned to University of Texas.

Also, the detection of breast or ovarian cancer based on the detection of mutated forms of the progesterone receptor gene has been reported (U.S. Pat. No. 5,683,885, issued Nov. 4, 1997, to Kieback, and U.S. Pat. No. 5,645,995 issued Jul. 8, 1997, both of which are assigned to Baylor College of Medicine.) Further, the use of the glycoprotein Mullerian Inhibiting Substance (MIS) as a target for treating certain tumors, including ovarian tumors, has been reported (See U.S. Pat. No. 5,661,126 issued Aug. 26, 1997 to Donahoe et al., and U.S. Pat. No. 5,547,856 issued Aug. 20, 1996, and assigned to General Hospital Corporation). Also, the use of CA125 as a target for ovarian cancer therapy has been reported. Particularly, AltaRex corporation has ongoing clinical trials involving their OvaRex monoclonal antibody which binds CA125.

However, notwithstanding what has been reported, there exists a significant need for the identification of novel gene targets for the treatment and diagnosis of ovarian cancer, especially given the huge human toll caused by this disease annually.

### SUMMARY OF THE INVENTION

The present invention provides nucleic acids and antigens encoded thereby for cancer treatment and diagnosis. Representative nucleic acids encoding cancer antigens include nucleic acids having (a) the nucleotide sequence of any one of SEQ ID NOs: 1, 2, 6, 9, 11, 14, 16, 20, 21, 23, 28, 37, 38, 39, 40, 41, 42, 43, and 44; (b) a nucleotide sequence encoding SEQ ID NO: 22 or 32; and (c) a nucleotide sequence complementary to (a) or (b). Nucleic acids of the invention also include nucleic acids having a sequence that is at least 70% identical or at least 90% identical to the sequence of the nucleic acid of claim 1, and which encodes a cancer cell antigen comprising one or more MHC class I binding epitopes. Additional nucleic acids of the invention encode cancer antigens comprising one or more MHC class I binding epitopes, wherein the nucleic acid hybridizes to the complement of the disclosed nucleic acids under the following stringent conditions: a final wash in 0.1×SSC at 65°.

Representative cancer antigens include (a) antigens encoded by a nucleic acid sequence of claim 1; and (b) fragments or variants of (a) that bind to antibodies that specifically bind the antigen of (a). Antibodies that specifically bind to the cancer antigens of the invention are also provided, including monoclonal antibodies and antigen binding fragments thereof. Useful monoclonal antibodies include chimeric, human, or humanized antibodies. In one embodiment of the invention, antibodies that specifically bind to the Anat-2 antigen are provided.

The disclosed nucleic acids, cancer antigens, and antibodies are useful for cancer diagnosis. For example, in representative embodiments the invention, the diagnosis involves detecting a nucleic acid in a cell sample using methods for hybridizing or amplifying the disclosed nucleic acid. In other representative embodiments of the invention, the diagnosis involves detecting a cancer antigen encoded by the disclosed nucleic acids, for example using an antibody that specifically binds to the antigen. Antibody detection methods include ELISA and competitive binding assays. Diagnostic reagents are also provided, which can comprise a disclosed nucleic acid or cancer antigen in combination with a detectable label. The diagnosis can comprise identifying a subject at risk for cancer based on elevated expression of the disclosed nucleic acid and cancer antigens.

Cancer antigens of the invention can include one or more MHC class I binding epitopes, including for example, an HLA-A0201 binding epitope, an HLA-24 binding epitope, an HLA-A3 binding epitope, an HLA-A1 binding epitope, an HLA-B7 binding epitope, and combinations thereof. The MHC class I binding epitopes mediate cytotoxic T cell lysis. Thus, the present invention also provides vaccines comprising the disclosed cancer antigens in combination with an adjuvant. Methods for treating cancer via administration of the vaccine are also provided.

Additionally provided therapeutic reagents, and methods for using the same, include (a) antisense oligonucleotides or ribozymes which hybridize to and may block expression of the disclosed nucleic acids; and (b) monoclonal antibodies and antigen binding fragments thereof, which bind to the disclosed cancer antigens. The therapeutic reagents can include an effector moiety, which is bound either directly or indirectly to the nucleic acid or antibody to be administered. Representative effector moieties include radionuclides, enzymes, cytotoxins, growth factors, and drugs. The disclosed cancer therapies can be used in combination with other cancer therapies, including chemotherapy.

### BRIEF DESCRIPTION OF THE DRAWINGS

FIG. 1 is an electronic Northern profile depicting the gene expression profile of this fragment as determined using the Gene Logic datasuite. The values along the y-axis represent expression intensities in Gene Logic units. Each blue circle on the figure represents an individual patient sample. The bar graph on the left of the figure depicts the percentage of each tissue type found to express the gene fragment.

FIG. 2(a) shows expression of Anat 2 in normal tissues, as determined using Clontech's human normal multiple tissue cDNA panel (MTC panel, catalog # K1421-1) Upper panel; Anat expression, lower panel; Glyceraldehyde 3-phosphate dehydrogenase (GAPDH) expression.

GAPDH is a housekeeping gene expressed at high levels in all human tissues and is used here as a control for cDNA integrity.

FIG. 2(b) shows expression in normal heart was next examined using Clontech's human cardiovascular multiple tissue cDNA panel (catalog # K1427-1).

FIG. 2(c) depicts Anat 2 expression in brain tissue using human brain cDNA panels from Biochain Institute (catalog #s 0516011 and 0516012).

FIG. 2(d) depicts Anat 2 expression in a panel of human ovarian tumor samples and 2 ovarian tumor cell lines. The ovarian tumor samples were obtained from the Cooperative Human Tissue Network (CHTN); the cell lines Ovcar-3 and PA1 were obtained from the ATCC. RNA was isolated from each sample and cell line using Qiagen's RNeasy kit (catalog # 75162).

FIG. 3 shows an electronic Northern profile for the EDG7 gene.

FIG. 4 shows the results of PCR experiments which measured EDG7 expression in normal human tissues.

FIG. 5 shows the results of PCR experiments which measured EDG7 expression in cardiovascular tissue.

FIG. 6 shows the results of PCR experiments that measured EDG7 expression in human ovarian tumor samples and cell lines.

FIG. 7 shows an immunoblot of total proteins (25 mg) from cell lysates (lanes 1, 3 and 5) or biotinylated proteins on streptavidin beads. This immunoblot shows the presence of biotinylated Anat-2 (lanes 2 and 4) indicating that the Anat-2 protein is expressed on the surface of the cells.

FIG. 8 shows the results of a typical Western that determined the expression of Anat-2 by transfected cell lines (lanes 1-8) relative to a non-transfected cell line (lane 9) control.

FIG. 9 shows an immunoblot comparing the expression of Anat-2 by 8 stable cell lines that express Anat-2 (lanes 1-8) relative to a positive control cell line that expresses B7.2 (lane 9).

FIG. 10(a) shows the results of an ELISA measuring the binding of antibodies to Anat-2 Ig compared to B7.1-Ig.

FIG. 10(b) shows the results of a FACS assay measuring the binding of Anat-2 specific antibodies to stable transfected Anat-2 CHO cells.

FIG. 11 shows the results of an immunoblot experiment that compared the binding of an anti-Anat-2 murine monoclonal antibody 6B8, to Anat-2 relative to Anat-3. This experiment shows that Anat-3 was not bound by 6B8.

FIG. 12 shows immunohistochemical data demonstrating surface binding of Anat-2 monoclonal antibody to an ovarian carcinoma cell.

FIG. 13 shows immunohistochemical data demonstrating the binding of Anat-2 murine monoclonal antibody 6B8 to ovarian tumor samples.

FIG. 14 shows an alignment of human MERET protein (SEQ ID NO: 22) and mouse MERET protein.

FIG. 15 is an electronic northern, which shows expression of MERET in the indicated tissues. MERET is highly upregulated in 73% of ovarian carcinomas.

FIG. 16 is a photograph of a gel showing the results of RT-PCR analysis in the indicated normal tissues. MERET is weakly expressed in normal testis and spleen. GAPDH was used as a control.

### DETAILED DESCRIPTION OF THE INVENTION

The present invention in part provides sequences of genes that are upregulated in ovarian cancer. These sequences (ESTs) were identified using the Gene Logic Gene Express Oncology DataSuite. Particularly, DataSuite analysis of gene expression in ovarian tumor tissue compared to mixed normal tissue (lung, liver, kidney, breast, pancreas, colon and ovary) indicated that genes identified infra, are upregulated greater than five-fold in the ovarian tumor samples as compared to the mixed normal tissue set.

In particular, the expression of these sequences is either absent or very low in normal tissues whereas expression in ovarian tumor tissues is very high. It has been found that of genes, (many) are expressed in >70% of the ovarian tumor samples analyzed. This high level of expression suggests that these genes or the corresponding protein antigen should be suitable targets for ovarian cancer therapy and diagnosis, or other cancers where these antigens are upregulated. In particular, these results suggest that these genes or antigens can be used to develop potential vaccine therapy, monoclonal antibodies, small molecule inhibitors, anti-sense therapies or ribozymes that target these genes or the corresponding proteins.

All of the genes identified herein are potentially useful targets for treatment and diagnosis of ovarian cancers, as well as other cancers and non-neoplastic cell growth disorders such as hyperplasia, metaplasia, and dysplasia. Thus, the disclosed genes and proteins may also be useful targets in cancers such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstroom's macroglobulinemia, and heavy chain disease.

The relative efficacy of the disclosed genes and proteins as targets for therapy and/or diagnosis, and the nature of the therapy or diagnosis, depends in part on the levels of expression and whether these proteins are expressed intracellularly or on the surface of tumor cells. In particular, surface proteins are appropriate targets for antibody-based therapies. As noted, antibody-based therapies are one embodiment of this invention. The antibodies are administered in naked form or conjugated to effector moieties e.g., radiolabels, therapeutic enzymes or drugs.

The present invention also provides novel gene targets which may be expressed in altered form in ovarian tumors, e.g. splice variants, that are overexpressed in ovarian tumors. The subject invention, in a less preferred embodiment, includes the synthesis of oligonucleotides having sequences in the antisense orientation relative to the genes identified by the present inventors which are upregulated by ovarian cancer tissues. Suitable therapeutic antisense oligonucleotides will typically vary in length from two to several hundred nucleotides in length, more typically about 50-70 nucleotides in length or shorter. These antisense oligonucleotides may be administered as naked DNAs or in protected forms, e.g., encapsulated in liposomes. The use of liposomal or other protected forms may be advantageous as it may enhance in vivo stability and delivery to target sites, i.e., ovarian tumor cells.

Also, the subject ovarian genes may be used to design novel ribozymes that target the cleavage of the corresponding mRNAs in ovarian tumor cells. Similarly, these ribozymes may be administered in free (naked) form or by the use of delivery systems that enhance stability and/or targeting, e.g., liposomes. Ribozymal and antisense therapies used to target genes that are selectively expressed by cancer cells are well known in the art.

Also, the present invention embraces the administration of use of DNAs that hybridize to the novel gene targets identified infra, attached to therapeutic effector moieties, e.g., radiolabels, e.g., yttrium, iodine, cytotoxins, cytotoxic enzymes, in order to selectively target and kill cells that express these genes, i.e., ovarian tumor cells.

Also, the present invention embraces the treatment and/or diagnosis of ovarian cancer by targeting altered genes or the corresponding altered protein, particularly splice variants that are expressed in altered form in ovarian cells. These methods will provide for the selective detection of cells and/or eradication of cells that express such altered forms thereby avoiding adverse effects to normal cells.

Still further, the present invention encompasses non-antibody protein based therapies.

Particularly, the invention encompasses the use of peptides or protein encoded by one of the novel cDNAs disclosed infra, or a fragment or variant thereof. It is anticipated that these antigens may be used as therapeutic or prophylactic anti-tumor vaccines. For example, a particular contemplated application of these antigens involves their administration with adjuvants that induce a cytotoxic T lymphocyte response. An especially preferred adjuvant developed by the Assignee of this application, IDEC Pharmaceuticals Corporation, is disclosed in U.S. Pat. Nos. 5,709,860, 5,695,770, and 5,585,103, the disclosures of which are incorporated by reference in their entirety. In particular, the use of this adjuvant to promote CTL responses against prostate and papillomavirus related human ovarian cancer has been suggested.

Also, administration of the subject ovarian antigens in combination with an adjuvant may result in a humoral immune response against such antigens, thereby delaying or preventing the development of ovarian cancer.

Essentially, these embodiments of the invention will comprise administration of one or both of the subject novel ovarian cancer antigens, ideally in combination with an adjuvant, e.g., PROVAXS, which comprises a microfluidized adjuvant containing Squalene, Tween and Pluronic, in an amount sufficient to be therapeutically or prophylactically effective. A typical dosage will range from 50 to 20,000 mg/kg body weight, have typically 100 to 5000 mg/kg body weight.

Alternatively, the subject ovarian tumor antigens may be administered with other adjuvants, e.g., ISCOMS, DETOX, SAF, Freund's adjuvant, Alum, Saponin, among others.

The preferred embodiment of the invention will comprise the preparation of monoclonal antibodies against the antigens encoded by the novel genes containing the nucleic acid sequences disclosed infra. Such monoclonal antibodies will be produced by conventional methods and include human monoclonal antibodies, humanized monoclonal antibodies, chimeric monoclonal antibodies, single chain antibodies, e.g., scFv's and antigen-binding antibody fragments such as Fabs, 2 Fabs, and Fab′ fragments. Methods for the preparation of monoclonal antibodies and fragments thereof, e.g., by pepsin or papain-mediated cleavage are well known in the art. In general, this will comprise immunization of an appropriate (non-homologous) host with the subject ovarian cancer antigens, isolation of immune cells therefrom, use of such immune cells to make hybridomas, and screening for monoclonal antibodies that specifically bind to either of such antigens.

These monoclonal antibodies and fragments will be useful for passive anti-tumor immunotherapy, or may be attached to therapeutic effector moieties, e.g., radiolabels, cytotoxins, therapeutic enzymes, agents that induce apoptosis, in order to provide for targeted cytotoxicity, i.e., killing of human ovarian tumor cells. Given the fact that the subject genes are apparently not significantly expressed by many normal tissues this should not result in significant adverse side effects (toxicity to non-target tissues).

In this embodiment, such antibodies or fragments will be administered in labeled or unlabeled form, alone or in combination with other therapeutics, e.g., chemotherapeutics such as progestin, EGFR, Taxol, etc. The administered composition will include a pharmaceutically acceptable carrier, and optionally adjuvants, stabilizers, etc., used in antibody compositions for therapeutic use.

Preferably, such monoclonal antibodies will bind the target antigens with high affinity, e.g., possess a binding affinity (Kd) on the order of 10-6 to 10-10 M.

As noted, the present invention also embraces diagnostic applications that provide for detection of the genes disclosed herein. Essentially, this will comprise detecting the expression of one or both of these genes at the DNA level or at the protein level.

At the DNA level, expression of the subject genes will be detected by known DNA detection methods, e.g., Northern blot hybridization, strand displacement amplification (SDA), catalytic hybridization amplification (CHA), and other known DNA detection methods.

Preferably, a cDNA library will be made from ovarian cells obtained from a subject to be tested for ovarian cancer by PCR using primers corresponding to either or both of the novel genes disclosed in this application.

The presence or absence of ovarian cancer will be determined based on whether PCR products are obtained, and the level of expression. The levels of expression of such PCR product may be quantified in order to determine the prognosis of a particular ovarian cancer patient (as the levels of expression of the PCR product likely will increase as the disease progresses.) This may provide a method of monitoring the status of an ovarian cancer patient. Of course, suitable controls will be effected.

Alternatively, the status of a subject to be tested for ovarian cancer may be evaluated by testing biological fluids, e.g., blood, urine, ovarian tissue, with an antibody or antibodies or fragment that specifically binds to the novel ovarian tumor antigens disclosed herein.

Methods for using antibodies to detect antigen expression are well known and include ELISA, competitive binding assays, etc. In general, such assays use an antibody or antibody fragment that specifically binds the target antigen directly or indirectly bound to a label that provides for detection, e.g., a radiolabel enzyme, fluorophore, etc.

Patients which test positive for the presence of the antigen on ovarian cells will be diagnosed as having or being at increased risk of developing ovarian cancer. Additionally, the levels of antigen expression may be useful in determining patient status, i.e., how far the disease has advanced (stage of ovarian cancer).

As noted, the present invention provides novel genes and corresponding antigens that correlate to human ovarian cancer. The present invention also embraces variants thereof. By “variants” is intended sequences that are at least 75% identical thereto, more preferably at least 85% identical, and most preferably at least 90% identical when these DNA sequences are aligned to the subject DNAs or a fragment thereof having a size of at least 50 nucleotides. This includes in particular allelic variants of the subject genes.

Also, the present invention provides for primer pairs that result in the amplification DNAs encoding the subject novel genes or a portion thereof in an mRNA library obtained from a desired cell source, typically human ovarian cell or tissue sample. Typically, such primers will be on the order of 12 to 50 nucleotides in length, and will be constructed such that they provide for amplification of the entire or most of the target gene.

Also, the invention embraces the antigens encoded by the subject DNAs or fragments thereof that bind to or elicit antibodies specific to the full length antigens. Typically, such fragments will be at least 10 amino acids in length, more typically at least 25 amino acids in length.

As noted, the subject genes are expressed in a majority of ovarian tumor samples tested.

The invention further contemplates the identification of other cancers that express such genes and the use thereof to detect and treat such cancers. For example, the subject genes or variants thereof may be expressed on other cancers, e.g., breast, pancreas, lung or colon cancers.

Essentially, the present invention embraces the detection of any cancer wherein the expression of the subject novel genes or variants thereof correlate to a cancer or an increased likelihood of cancer. In order to better describe the invention, the following definitions are provided.

Otherwise, all terms have their ordinary meaning as they would be construed by one skilled in the art.

“Isolated tumor antigen or tumor protein” refers to any protein that is not in its normal cellular millieu. This includes by way of example compositions comprising recombinant proteins encoded by the genes disclosed infra, pharmaceutical compositions comprising such purified proteins, diagnostic compositions comprising such purified proteins, and isolated protein compositions comprising such proteins. In preferred embodiments, an isolated ovarian tumor protein according to the invention will comprise a substantially pure protein, i.e., a protein that it is substantially free of other proteins, preferably that is at least 90% pure, that comprises the amino acid sequence contained herein or natural homologues or mutants having essentially the same sequence. A naturally occurring mutant might be found, for instance, in tumor cells expressing a gene encoding a mutated protein according to the invention.

“Native tumor antigen or tumor protein” refers to a protein that is a non-human primate homologue of the protein having the amino acid sequence contained infra.

“Isolated ovarian tumor gene or nucleic acid sequence” refers to a nucleic acid molecule that encodes a tumor antigen according to the invention which is not in its normal human cellular millieu, e.g., is not comprised in the human or non-human primate chromosomal DNA. This includes by way of example vectors that comprise a gene according to the invention, a probe that comprises a gene according to the invention, and a nucleic acid sequence directly or indirectly attached to a detectable moiety, e.g. a fluorescent or radioactive label, or a DNA fusion that comprises a nucleic acid molecule encoding a gene according to the invention fused at its 5′ or 3′ end to a different DNA, e.g. a promoter or a DNA encoding a detectable marker or effector moiety. Also included are natural homologues or mutants having substantially the same sequence. Naturally occurring homologies that are degenerate would encode the same protein including nucleotide differences that do not change the corresponding amino acid sequence.

Naturally occurring mutants might be found in tumor cells, wherein such nucleotide differences may result in a mutant tumor antigen. Naturally occurring homologues containing conservative substitutions are also encompassed.

“Variant of ovarian tumor antigen or tumor protein” refers to a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to the corresponding native tumor antigen wherein sequence identity is as defined infra. Preferably, this variant will possess at least one biological property in common with the native protein.

“Variant of ovarian tumor gene or nucleic acid molecule or sequence” refers to a nucleic acid sequence that possesses at least 90% sequence identity, more preferably at least 91%, more preferably at least 92%, even more preferably at least 93%, still more preferably at least 94%, even more preferably at least 95%, still more preferably at least 96%, even more preferably at least 97%, even more preferably at least 98% sequence identity, and most preferably at least 99% sequence identity, to the corresponding native human nucleic acid sequence, wherein “sequence identity” is as defined infra.

“Fragment of ovarian antigen encoding nucleic acid molecule or sequence” refers to a nucleic acid sequence corresponding to a portion of the native human gene wherein said portion is at least about 50 nucleotides in length, or 100, more preferably at least 200 or 300 nucleotides in length.

“Antigenic fragments of ovarian tumor antigen” refer to polypeptides corresponding to a fragment of an ovarian protein or a variant or homologue thereof that when used itself or attached to an immunogenic carrier that elicits antibodies that specifically bind the protein. Typically such antigenic fragments will be at least 20 amino acids in length.

Sequence identity or percent identity is intended to mean the percentage of the same residues shared between two sequences, when the two sequences are aligned using the Clustal method [Higgins et al, Cabios 8: 189-191 (1992)] of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3 window value in pairwise alignment=5; diagonals saved in pairwise alignment=5. The residue weight table used for the alignment program is PAM250 [Dayhoff et al., in Atlas of Protein Sequence and Structure, Dayhoff, Ed., NDRF, Washington, Vol. 5, suppl. 3, p. 345, (1978)].

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to the unmodified human gene determining percent conservation with e.g., a non-human gene, a murine gene homolog, when determining percent conservation. Conservative amino acid changes satisfying this requirement are: R-K; E-D, Y-F, L-M; V-I, Q-H.

The invention provides polypeptide fragments of the disclosed proteins. Polypeptide fragments of the invention can comprise at least 8, more preferably at least 25, still more preferably at least 50 amino acid residues of the protein or an analogue thereof. More particularly such fragment will comprise at least 75, 100, 125, 150, 175, 200, 225, 250, 275 residues of the polypeptide encoded by the corresponding gene. Even more preferably, the protein fragment will comprise the majority of the native protein, e.g. about 100 contiguous residues of the native protein.

The invention also encompasses mutants of the novel ovarian proteins disclosed infra which comprise an amino acid sequence that is at least 80%, more preferably 90%, still more preferably 95-99% similar to the native protein.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant.

Protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the gene are also variants. Covalent variants can be prepared by linking functionalities to groups which are found in the amino acid chain or at the N- or C-terminal residue, as is known in the art.

It will be recognized in the art that some amino acid sequence of the ovarian proteins of the invention can be varied without significant effect on the structure or function of the protein.

If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361: 266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the ovarian proteins disclosed infra which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247: 1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2: 331-340 (1967); Robbins et al., Diabetes 36: 838-845 (1987); Cleland et al., Crit. Rev. Therapeutic Drug Carrier Systems 10: 307-377 (1993)).

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., JMol. Biol. 224: 899-904 (1992) and de Vos et al. Science 255: 306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

Fusion proteins comprising proteins or polypeptide fragments of the subject ovarian tumor antigen can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of a protein according to the invention or a fragment thereof can be used to target other protein domains to cellular locations in which the domains are not normally found, such as bound to a cellular membrane or secreted extracellularly. A fusion protein comprises two protein segments fused together by means of a peptide bond. Amino acid sequences for use in fusion proteins of the invention can utilize the amino acid sequence disclosed herein or proteins encoded by the nucleic acid sequences disclosed infra.

The second protein segment can be a full-length protein or a polypeptide fragment.

Proteins commonly used in fusion protein construction include B-galactosidase, B-glucuronidase, green fluorescent protein (GFP), autofluorescent proteins, including blue fluorescent protein (BFP), glutathione-5-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence encoding an amino acid sequence corresponding to an ovarian antigen of the invention, e.g., Anat-2, in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a sequence encoding the protein can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein.

Such covalent attachments can be made using known chemical or enzymatic methods.

A protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as mice, rats, guinea pigs, cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983).

Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of the nucleic acid sequences provided herein. Polynucleotde probes of the invention can comprise a detectable label, such as a radioisotopic, fluorescent, enzymatic, or chemiluminescent label.

Isolated genes corresponding to the cDNA sequences disclosed herein are also provided.

Standard molecular biology methods can be used to isolate the corresponding genes using the cDNA sequences provided herein. These methods include preparation of probes or primers from the nucleotide sequence disclosed herein for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art.

Polynucleotide molecules can be on linear or circular molecules. They can be on autonomously replicating molecules or on molecules without replication sequences. They can be regulated by their own or by other regulatory sequences, as is known in the art.

The synthesis of antibodies that bind ovarian antigens according to the invention will be effected by well known methods. For examples, monoclonal antibodies that bind ovarian antigens disclosed infra, e.g., Anat-2, having desirable properties will be derived, cells that express these monoclonal antibodies isolated, and these cells used to make hybridomas or alternatively these cells used to isolate the corresponding antibody genes, and these genes used to produce the corresponding antibody by recombinant methods. Oligonucleotide synthesis techniques compatible with this aspect of the invention are well known to the skilled artisan and may be carried out using any of several commercially available automated synthesizers. In addition, DNA sequences encoding several types of heavy and light chains set forth herein can be obtained through the services of commercial DNA synthesis vendors. The genetic material obtained using any of the foregoing methods may then be altered or modified to provide antibodies compatible with the present invention.

A variety of different types of antibodies may be expressed according to the instant invention. “Antibodies” refers to such assemblies which have significant known specific immunoreactive activity to an antigen (i.e., an ovarian associated antigen), comprising light and heavy chains, with or without covalent linkage between them. “Modified antibodies” according to the present invention are held to mean immunoglobulins, antibodies, or immunoreactive fragments or recombinants thereof, in which at least a fraction of one or more of the constant region domains has been deleted or otherwise altered so as to provide desired biochemical characteristics such as the ability to non-covalently dimerize, increased tumor localization or reduced serum half-life when compared with a whole, unaltered antibody of approximately the same immunogenicity. For the purposes of the instant application, immunoreactive single chain antibody constructs having altered or omitted constant region domains may be considered to be modified antibodies. As discussed above, preferred modified antibodies or domain deleted antibodies expressed using the polycistronic system of the present invention have at least a portion of one of the constant domains deleted. More preferably, one entire domain of the constant region of the modified antibody will be deleted and even more preferably the entire CH2 domain will be deleted.

Basic immunoglobulin structures in vertebrate systems are relatively well understood. As will be discussed in more detail below, the generic term “immunoglobulin” comprises five distinct classes of antibody that can be distinguished biochemically. While all five classes are clearly within the scope of the present invention, the following discussion will generally be directed to the class of IgG molecules. With regard to IgG, immunoglobulins comprise two identical light polypeptide chains of molecular weight approximately 23,000 Daltons, and two identical heavy chains of molecular weight 53,000. The four chains are joined by disulfide bonds in a “Y” configuration wherein the light chains bracket the heavy chains starting at the mouth of the “Y” and continuing through the variable region.

More specifically, both the light and heavy chains are divided into regions of structural and functional homology. The terms “constant” and “variable” are used functionally. In this regard, it will be appreciated that the variable domains of both the light (VL) and heavy (VH) chains determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like.

By convention the numbering of the constant region domains increases as they become more distal from the antigen binding site or amino-terminus of the antibody. Thus, the CH3 and CL domains actually comprise the carboxy-terminus of the heavy and light chains respectively.

Light chains are classified as either kappa or lambda (K, B). Each heavy chain class may be bound with either a kappa or lambda light chain. In general, the light and heavy chains are covalently bonded to each other, and the “tail” portions of the two heavy chains are bonded to each other by covalent disulfide linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. At the N-terminus is a variable region and at the C-terminus is a constant region. Those skilled in the art will appreciate that heavy chains are classified as gamma, mu, alpha, delta, or epsilon, (y, a, 8, s) with some subclasses among them. It is the nature of this chain that determines the “class” of the antibody as IgA, IgD, IgE IgG, or IgM. The immunoglobulin subclasses (isotypes) e.g. IgG), IgG2, IgG3, IgG4, IgA), etc. are well characterized and are known to confer functional specialization. Modified versions of each of these classes and isotypes are readily discernable to the skilled artisan in view of the instant disclosure and, accordingly, are within the purview of the instant invention.

As indicated above, the variable region allows the antibody to selectively recognize and specifically bind epitopes on immunoreactive antigens. That is, the VL domain and VH domain of an antibody combine to form the variable region that defines a three dimensional antigen binding site. This quaternary antibody structure provides for an antigen binding site present at the end of each arm of the Y. More specifically, the antigen binding site is defined by three complementary determining regions (CDRs) on each of the VH and VL chains.

The six CDRs present on each monomeric antibody (H2L2) are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a p-sheet conformation and the CDRs form loops connecting, and in some cases forming part of, the β-sheet structure. Thus, these framework regions act to form a scaffold that provides for positioning the six CDRs in correct orientation by inter-chain, non-covalent interactions. In any event, the antigen binding site formed by the positioned CDRs defines a surface complementary to the epitope on the immunoreactive antigen. This complementary surface promotes the non-covalent binding of the antibody to the immunoreactive antigen epitope.

For the purposes of the present invention, it should be appreciated that modified antibodies capable of forming functional antibodies may comprise any type of variable region that provides for the association of the resultant antibody with the selected antigen. In this regard, the variable region may comprise or be derived from any type of mammal that can be induced to mount a humoral response and generate immunoglobulins against the desired antigen.

As such, the variable region of the modified antibodies maybe, for example, of human, murine, non-human primate (e.g. cynomolgus monkeys, macaques, etc.) or lupine origin. In particularly preferred embodiments both the variable and constant regions of compatible modified antibodies are human. In other selected embodiments the variable regions of compatible antibodies (usually derived from a non-human source) may be engineered or specifically tailored to improve the binding properties or reduce the immunogenicity of the molecule. In this respect, variable regions useful in the present invention may be humanized or otherwise altered through the inclusion of imported DNA or amino acid sequences. For the purposes of the instant application the term “humanized antibody” shall mean an antibody derived from a non-human antibody, typically a murine antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693, 762 all of which are hereby incorporated by reference in their entirety.

Those skilled in the art will appreciate that the technique set forth in option (a) above will produce “classic” chimeric antibodies. In the context of the present application the term “chimeric antibodies” will be held to mean any antibody wherein the immunoreactive region or site is obtained or derived from a first species and the constant region (which may be intact, partial or modified in accordance with the instant invention) is obtained from a second species.

In preferred embodiments the antigen binding region or site will be from a non-human source (e.g. mouse) and the constant region is human. While the immunogenic specificity of the variable region is not generally affected by its source, a human constant region is less likely to elicit an immune response from a human subject than would the constant region from a non-human source.

Preferably, the variable domains in both the heavy and light chains are altered by at least partial replacement of one or more CDRs and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. It must be emphasized that it may not be necessary to replace all of the CDRs with the complete CDRs from the donor variable region to transfer the antigen binding capacity of one variable domain to another. Rather, it may only be necessary to transfer those residues that are necessary to maintain the activity of the antigen binding site.

Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that modified antibodies compatible with the instant invention will comprise antibodies, or immunoreactive fragments thereof, in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In preferred embodiments, the constant region of the modified antibodies will comprise a human constant region. Modifications to the constant region compatible with the instant invention comprise additions, deletions or substitutions of one or more amino acids in one or more domains. That is, the modified antibodies disclosed herein may comprise alterations or modfications to one or more of the three heavy chain constant domains (CH1, CH2 or CH3) and/or to the light chain constant domain (CL). As will be discussed in more detail below and shown in the examples, preferred embodiments of the invention comprise modified constant regions wherein one or more domains are partially or entirely deleted (“domain deleted antibodies”). In especially preferred embodiments compatible modified antibodies will comprise domain deleted constructs or variants wherein the entire CH2 domain has been removed (ACH2 constructs). For other preferred embodiments a short amino acid spacer may be substituted for the deleted domain to provide flexibility and freedom of movement for the variable region.

As previously indicated, the subunit structures and three dimensional configuration of the constant regions of the various immunoglobulin classes are well known. For example, the CH2 domain of a human IgG Fc region usually extends from about residue 231 to residue 340 using conventional numbering schemes. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues while the hinge region of an IgG molecule joins the CH2 domain with the CH1 domain. This hinge region encompasses on the order of 25 residues and is flexible, thereby allowing the two N-terminal antigen binding regions to move independently.

Besides their configuration, it is known in the art that the constant region mediates several effector functions. For example, binding of the Cl component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. Although various Fc receptors and receptor sites have been studied to a certain extent, there is still much which is unknown about their location, structure and functioning.

As discussed above, the modification of the constant region as described herein allows the disclosed modified antibodies to spontaneously assemble or associate into stable dimeric constructs or tetravalent antibodies. Moreover, while not limiting the scope of the present invention, it is believed that antibodies comprising constant regions modified as described herein provide for altered effector functions that, in turn, affect the biological profile of the administered antibody. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate compliment binding and thus reduce the serum half life and nonspecific association of a conjugated cytotoxin. Yet other modifications of the constant region may be used to eliminate disulfide linkages or oligosaccharide moities that allow for enhanced localization due to increased antigen specificity or antibody flexibility. More generally, those skilled in the art will realize that antibodies modified as described herein may exert a number of subtle effects that may or may not be readily appreciated. However the resulting physiological profile, bioavailability and other biochemical effects of the modifications, such as tumor localization and serum half-life, may easily be measured and quantified using well know immunological techniques without undue experimentation.

Similarly, modifications to the constant region in accordance with the instant invention may easily be made using well known biochemical or molecular engineering techniques well within the purview of the skilled artisan. In this respect the examples appended hereto provide various constructs having constant regions modified in accordance with the present invention.

More specifically, the exemplified constructs comprise chimeric and humanized antibodies having human constant regions that have been engineered to delete the CH2 domain. Those skilled in the art will appreciate that such constructs are particularly preferred due to the regulatory properties of the CH2 domain on the catabolic rate of the antibody.

Besides the deletion of whole constant region domains, it will be appreciated that antibody constructs of the present invention may be provided by the partial deletion or substitution of a few or even a single amino acid as long as it permits the desired non-covalent association between the antibody and targeted ovarian antigen. For example, the mutation of a single amino acid in selected areas of the CH2 domain may be enough to substantially reduce Fc binding and thereby increase tumor localization. Similarly, it may be desirable to simply delete that part of one or more constant region domains that control the effector function (e.g. complement CLQ binding) to be modulated.

Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other preferred embodiments may comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

Following manipulation of the isolated genetic material to provide modified antibodies as set forth above, the genes are typically inserted in an expression vector for introduction into host cells that may be used to produce the desired quantity of antibody.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Polynucleotide molecules comprising the coding sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Also included are polynucleotide molecules comprising the promoter and UTR sequences of the subject novel genes, operably linked to the associated protein coding sequence and/or other sequences encoding a detectable or selectable marker. Such promoter and/or UTR-based constructs are useful for studying the transcriptional and translational regulation of protein expression, and for identifying activating and/or inhibitory regulatory proteins.

An expression construct can be introduced into a host cell. The host cell comprising the expression construct can be any suitable prokaryotic or eukaryotic cell. Expression systems in bacteria include those described in Chang et al., Nature 275: 615 (1978); Goeddel et al., Nature 281: 544 (1979); Goeddel et al., NucleicAcidsRes. 8: 4057 (1980); EP 36,776; U.S. Pat. No. 4,551,433; deBoer et al., Proc. Natl. Acad. Sci. USA 80: 21-25 (1983); and Siebenlist et al., Cell 20: 269 (1980).

Expression systems in yeast include those described in Hinnnen et al., Proc. Natl. Acad. Sci. USA 75: 1929 (1978); Ito et al., J Bacteriol153: 163 (1983); Kurtz et al., Mol. Cell. Biol. 6: 142 (1986); Kunze et al., JBasic Microbiol. 25: 141 (1985); Gleeson et al., J. Gen. Microbiol. 132: 3459 (1986), Roggenkamp et al., Mol. Gen. Genet. 202: 302 (1986)); Das et al., J. Bacteriol. 158: 1165 (1984); De Louvencourt et al., J. Bacteriol. 154: 737 (1983), Van den Berg et al., BiolTechnology 8: 135 (1990); Kunze et al., J. Basic Microbiol. 25: 141 (1985); Cregg et al., Mol. Cell. Biol. 5: 3376 (1985); U.S. Pat. No. 4,837,148; U.S. Pat. No. 4,929,555; Beach and Nurse, Nature 300: 706 (1981); Davidow et al., Curr. Genet. 10: 380 (1985); Gaillardin et al., Curr. Genet. 10: 49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475479 (1985); EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al., Ann. Rev. Microbiol. 42: 177 (1988); Carbonell et al., Gene 73: 409 (1988); Maeda et al., Nature 315: 592-594 (1985); Lebacq-Verheyden et al., Mol. Cell Biol. 8: 3129 (1988); Smith et al., Proc. Natl. Acad. Sci. USA 82: 8404 (1985); Miyajima et al., Gene 58: 273 (1987); and Martin et al., DNA 7: 99 (1988).

Numerous baculoviral strains and variants and corresponding permissive insect host cells from hosts are described in Luckow et al., BiolTechnology (1988) 6: 47-55, Miller et al., in GENETIC ENGINEERING (Setlow, J. K. et al. eds.), Vol. 8, pp. 277-279 (Plenum Publishing, 1986); and Maeda et al., Nature, 315: 592-594 (1985).

Mammalian expression can be accomplished as described in Dijkema et al., EMBO J. 4: 761 (1985); Gorman et al., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979); Barnes and Sato, Anal. Biochem. 102: 255 (1980); U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides of the nucleotide sequence shown in the figures herein. The transcription unit is located upstream to a coding sequence of the endogenous gene. The exogenous regulatory sequence directs transcription of the coding sequence of the endogenous gene.

The invention can also include hybrid and modified forms thereof including fusion proteins, fragments and hybrid and modified forms in which certain amino acids have been deleted or replaced, modifications such as where one or more amino acids have been changed to a modified amino acid or unusual amino acid.

Also included within the meaning of substantially homologous is any human or non-human primate protein which may be isolated by virtue of cross-reactivity with antibodies to proteins encoded by a gene described herein or whose encoding nucleotide sequences including genomic DNA, mRNA or cDNA may be isolated through hybridization with the complementary sequence of genomic or subgenomic nucleotide sequences or cDNA of a gene herein or fragments thereof. It will also be appreciated by one skilled in the art that degenerate DNA sequences can encode a tumor protein according to the invention and these are also intended to be included within the present invention as are allelic variants of the subject genes.

Preferred is an ovarian protein according to the invention prepared by recombinant DNA technology. By “pure form” or “purified form” or “substantially purified form” it is meant that a protein composition is substantially free of other proteins which are not the desired protein.

The present invention also includes therapeutic or pharmaceutical compositions comprising a protein according to the invention in an effective amount for treating patients with disease, and a method comprising administering a therapeutically effective amount of the protein.

These compositions and methods are useful for treating cancers associated with the subject proteins, e.g. ovarian cancer. One skilled in the art can readily use a variety of assays known in the art to determine whether the protein would be useful in promoting survival or functioning in a particular cell type.

In certain circumstances, it may be desirable to modulate or decrease the amount of the protein expressed by a cell, e.g. ovary cell. Thus, in another aspect of the present invention, anti-sense oligonucleotides can be made and a method utilized for diminishing the level of expression an ovarian antigen according to the invention by a cell comprising administering one or more anti-sense oligonucleotides. By anti-sense oligonucleotides reference is made to oligonucleotides that have a nucleotide sequence that interacts through base pairing with a specific complementary nucleic acid sequence involved in the expression of the target such that the expression of the gene is reduced. Preferably, the specific nucleic acid sequence involved in the expression of the gene is a genomic DNA molecule or mRNA molecule that encodes the gene. This genomic DNA molecule can comprise regulatory regions of the gene, or the coding sequence for the mature gene.

The term complementary to a nucleotide sequence in the context of antisense oligonucleotides and methods therefore means sufficiently complementary to such a sequence as to allow hybridization to that sequence in a cell, i.e., under physiological conditions. Antisense oligonucleotides preferably comprise a sequence containing from about 8 to about 100 nucleotides and more preferably the antisense oligonucleotides comprise from about 15 to about 30 nucleotides. Antisense oligonucleotides can also contain a variety of modifications that confer resistance to nucleolytic degradation such as, for example, modified internucleoside lineages [Uhlmann and Peyman, Chemical Reviews 90: 543-548 (1990); Schneider and Banner, Tetrahedron Lett. 31: 335, (1990) which are incorporated by reference], modified nucleic acid bases as disclosed in U.S. Pat. No. 5,958,773 and patents disclosed therein, and/or sugars and the like.

Any modifications or variations of the antisense molecule which are known in the art to be broadly applicable to antisense technology are included within the scope of the invention.

Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. [Branch, A. D., T. B. S. 23: 45-50 (1998)].

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

Additionally, the subject ovarian tumor proteins can also be linked or conjugated with agents that provide desirable pharmaceutical or pharmacodynamic properties. For example, the protein can be coupled to any substance known in the art to promote penetration or transport across the blood-brain barrier such as an antibody to the transferrin receptor, and administered by intravenous injection (see, for example, Friden et al., Science 259: 373-377 (1993) which is incorporated by reference). Furthermore, the subject ovarian protein can be stably linked to a polymer such as polyethylene glycol to obtain desirable properties of solubility, stability, half-life and other pharmaceutically advantageous properties. [See, for example, Davis et al., Enzyme Eng. 4: 169-73 (1978); Buruham, Am. J. Hosp. Pharm. 51: 210-218 (1994) which are incorporated by reference].

The compositions are usually employed in the form of pharmaceutical preparations. Such preparations are made in a manner well known in the pharmaceutical art. See, e.g. Remington Pharmaceutical Science, 18th Ed., Merck Publishing Co. Eastern PA, (1990). One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The subject ovarian protein, fragment or variant thereof can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing the subject ovarian proteins or variant or fragment thereof are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, the protein may be therapeutically administered by implanting into patients vectors or cells capable of producing a biologically-active form of the protein or a precursor of protein, i.e., a molecule that can be readily converted to a biological-active form of the protein by the body. In one approach, cells that secrete the protein may be encapsulated into semipermeable membranes for implantation into a patient. The cells can be cells that normally express the protein or a precursor thereof or the cells can be transformed to express the protein or a precursor thereof. It is preferred that the cell be of human origin and that the protein be a human protein when the patient is human. However, it is anticipated that non-human primate homologues of the protein discussed infra may also be effective.

In a number of circumstances it would be desirable to determine the levels of protein or corresponding mRNA in a patient. Evidence disclosed infra suggests the subject ovarian proteins may be expressed at different levels during some diseases, e.g., cancers, provides the basis for the conclusion that the presence of these proteins serves a normal physiological function related to cell growth and survival. Endogenously produced protein according to the invention may also play a role in certain disease conditions.

The term “detection” as used herein in the context of detecting the presence of protein in a patient is intended to include the determining of the amount of protein or the ability to express an amount of protein in a patient, the estimation of prognosis in terms of probable outcome of a disease and prospect for recovery, the monitoring of the protein levels over a period of time as a measure of status of the condition, and the monitoring of protein levels for determining a preferred therapeutic regimen for the patient, e.g. one with ovarian cancer.

To detect the presence of an ovarian protein according to the invention in a patient, a sample is obtained from the patient. The sample can be a tissue biopsy sample or a sample of blood, plasma, serum, CSF or the like. It has been found that the subject proteins are expressed at high levels in some cancers. Samples for detecting protein can be taken from ovarian tissues.

When assessing peripheral levels of protein, it is preferred that the sample be a sample of blood, plasma or serum. When assessing the levels of protein in the central nervous system a preferred sample is a sample obtained from cerebrospinal fluid or neural tissue.

In some instances, it is desirable to determine whether the gene is intact in the patient or in a tissue or cell line within the patient. By an intact gene, it is meant that there are no alterations in the gene such as point mutations, deletions, insertions, chromosomal breakage, chromosomal rearrangements and the like wherein such alteration might alter production of the corresponding protein or alter its biological activity, stability or the like to lead to disease processes. Thus, in one embodiment of the present invention a method is provided for detecting and characterizing any alterations in the gene. The method comprises providing an oligonucleotide that contains the gene, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize specifically to the gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, TaqI and AluI. Using the Southern blot protocol, which is well known in the art, this assay determines whether a patient or a particular tissue in a patient has an intact A or B gene or an A or B gene abnormality.

Hybridization to a gene would involve denaturing the chromosomal DNA to obtain a single-stranded DNA; contacting the single-stranded DNA with a gene probe associated with the gene sequence; and identifying the hybridized DNA-probe to detect chromosomal DNA containing at least a portion of a gene.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

A gene according to the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

Gene abnormalities can also be detected by using the PCR method and primers that flank or lie within the gene. The PCR method is well known in the art. Briefly, this method is performed using two oligonucleotide primers which are capable of hybridizing to the nucleic acid sequences flanking a target sequence that lies within a gene and amplifying the target sequence.

The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising the gene or a fragment thereof is then directly sequenced and analyzed by comparison of the sequence with the sequences disclosed herein to identify alterations which might change activity or expression levels or the like.

In another embodiment, a method for detecting a tumor protein according to the invention is provided based upon an analysis of tissue expressing the gene. Certain tissues such as ovarian tissues have been found to overexpress the subject gene. The method comprises hybridizing a polynucleotide to mRNA from a sample of tissue that normally expresses the gene. The sample is obtained from a patient suspected of having an abnormality in the gene.

To detect the presence of mRNA encoding the protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding the protein or a derivative of the cDNA as a probe, high stringency conditions can be used in order to prevent false positives, that is the hybridization and apparent detection of the gene nucleotide sequence when in fact an intact and functioning gene is not present. When using sequences derived from the gene cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. [Sambrook et al. (1989), supra].

In order to increase the sensitivity of the detection in a sample of mRNA encoding an ovarian protein according to the invention technique of reverse transcription/polymerization chain reaction (RT/PCR) can be used to amplify cDNA transcribed from mRNA encoding the protein. The method of RT/PCR is well known in the art, and can be performed as follows.

Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo (dT) sequence. The cDNA thus produced is then amplified using the PCR method and gene A or gene B specific primers. [Belyavsky et al., Nucl. Acid Res. 17: 2919-2932 (1989); Krug and Berger, Methods in Enzymology, 152: 316-325, Academic Press, NY (1987) which are incorporated by reference].

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. [Basic and Clinical Immunology, 217-262, Sites and Terr, eds., Appleton & Lange, Norwalk, Conn., (1991), which is incorporated by reference]. Preferred are binder-ligand immunoassay methods including reacting antibodies with an epitope or epitopes of the gene and competitively displacing a labeled gene encoding the protein or derivative thereof.

As used herein, a derivative of an ovarian protein according to the invention is intended to include a polypeptide in which certain amino acids have been deleted or replaced or changed to modified or unusual amino acids wherein the derivative is biologically equivalent to gene and wherein the polypeptide derivative cross-reacts with antibodies raised against the protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Polyclonal or monoclonal antibodies to the subject protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, typically a rabbit, hamster or a mouse.

Oligopeptides can be selected as candidates for the production of an antibody to the protein based upon the oligopeptides lying in hydrophilic regions, which are thus likely to be exposed in the mature protein. Suitable additional oligopeptides can be determined using, for example, the Antigenicity Index, Welling, G. W. et al., FEBS Lett. 188: 215-218 (1985), incorporated herein by reference.

As noted, a preferred aspect of the invention will comprise the administration of antibodies that target ovarian antigens identified infra, for the treatment of cancers wherein these antigens are upregulated, particularly ovarian cancers. These antibodies will be formulated and administered by conventional means for use of therapeutic antibodies for cancer treatment.

In preferred embodiments of the present invention, humanized monoclonal antibodies are provided, wherein the antibodies are specific for an ovarian protein according to the invention.

As defined previously, the phrase “humanized antibody” refers to an antibody derived from a non-human antibody, typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321: 522-525 (1986); Morrison et al., Proc. Natl. Acad. Sci, US. A., 81: 6851-6855 (1984); Morrison and Oi, Adv. Immunol., 44: 65-92 (1988); Verhoeyer et al., Science 239: 1534-1536 (1988); Padlan, Molec. Immun. 28: 489-498 (1991); Padlan, Molec. Immunol. 31 (3): 169-217 (1994); and Kettleborough, C. A. et al., Protein Eng. 4 (7): 773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196: 901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which patents are incorporated herein by reference.

Humanized antibodies to the subject ovarian tumor proteins can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNF, human CD4, L-selectin, gp39, and tetanus toxin. The monoclonal antibodies can be tested for the ability to inhibit or neutralize the biological activity or physiological effect of the corresponding protein.

WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, an ovarian protein or variants thereof according to the invention are used to immunize a transgenic animal as described above. Monoclonal antibodies are made using methods known in the art, and the specificity of the antibodies is tested using isolated protein.

Methods for preparation of the subject tumor proteins include, but are not limited to chemical synthesis, recombinant DNA techniques or isolation from biological samples.

Chemical synthesis of a peptide can be performed, for example, by the classical Merrifeld method of solid phase peptide synthesis (Merrifeld, J. Am. Chem. Soc. 85: 2149, 1963 which is incorporated by reference) or the FMOC strategy on a Rapid Automated Multiple Peptide Synthesis system [E. I. du Pont de Nemours Company, Wilmington, Del.) (Caprino and Han, J. Org. Chem. 37: 3404 (1972) which is incorporated by reference].

Polyclonal antibodies can be prepared by immunizing rabbits or other animals by injecting antigen followed by subsequent boosts at appropriate intervals. The animals are bled and sera assayed against purified protein usually by ELISA or by bioassay based upon the ability to block the action of the corresponding gene. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. [Milstein and Kohler, Nature 256: 495-497 (1975); Gulfre and Milstein, Methods in Enzymology: Immunochemical Techniques 73: 1-46, Langone and Banatis eds., Academic Press, (1981) which are incorporated by reference]. The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, this aspect of the present invention provides for a method for preventing or treating diseases involving overexpression of the protein by treatment of a patient with specific antibodies to the protein.

Specific antibodies, either polyclonal or monoclonal, to the protein can be produced by any suitable method known in the art as discussed above. For example, murine or human monoclonal antibodies can be produced by hybridoma technology or, alternatively, the protein, or an immunologically active fragment thereof, or an anti-idiotypic antibody, or fragment thereof can be administered to an animal to elicit the production of antibodies capable of recognizing and binding to the protein. Such antibodies can be from any class of antibodies including, but not limited to IgG, IgA, 1 gM, IgD, and IgE or in the case of avian species, IgY and from any subclass of antibodies.

Regardless of how clinically useful quantities are obtained, those used in the therapeutic methods of the present invention may be used in any one of a number of conjugated (i.e. an immunoconjugate) or unconjugated forms. Alternatively, the antibodies of the instant invention may be used in a nonconjugated or original form to harness the subject's natural defense mechanisms to eliminate the malignant cells. In particularly preferred embodiments, the antibodies may be conjugated to radioisotopes, such as 90Y, 125I, 131I, 123I, 111In, 105Rh, 153Sm, 67Cu, 67Ga, 166Ho, 177Lu, 186Re and 188Re using anyone of a number of well known chelators or direct labeling. In other embodiments, the disclosed compositions may comprise antibodies coupled to drugs, prodrugs or biological response modifiers such as methotrexate, adriamycin, and lymphokines such as interferon. Still other embodiments of the present invention comprise the use of antibodies conjugated to specific biotoxins such as ricin or diptheria toxin. In yet other embodiments the modified antibodies may be complexed with other immunologically active ligands (e.g. antibodies or fragments thereof) wherein the resulting molecule binds to both the neoplastic cell and an effector cell such as a T cell. The selection of which conjugated or unconjugated modified antibody to use will depend of the type and stage of cancer, use of adjunct treatment (e.g., chemotherapy or external radiation) and patient condition. It will be appreciated that one skilled in the art could readily make such a selection in view of the teachings herein.

As used herein, “a cytotoxin or cytotoxic agent” means any agent that is detrimental to the growth and proliferation of cells and may act to reduce, inhibit or destroy a cell or malignancy when exposed thereto. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. As will be discussed in more detail below, radionuclide cytotoxins are particularly preferred for use in the instant invention. However, any cytotoxin that acts to retard or slow the growth of immunoreactive cells or malignant cells or to eliminate these cells and may be associated with the antibodies disclosed herein is within the purview of the present invention.

It will be appreciated that, in previous studies, anti-tumor antibodies labeled with these isotopes have been used successfully to destroy cells in solid tumors in animal models, and in some cases in humans. The radionuclides act by producing ionizing radiation which causes multiple strand breaks in nuclear DNA, leading to cell death. The isotopes used to produce therapeutic conjugates typically produce high energy a- or (3-particles which have a short path length. Such radionuclides kill cells to which they are in close proximity, for example neoplastic cells to which the conjugate has attached or has entered. They have little or no effect on non-localized cells. Radionuclides are essentially non-immunogenic.

It will be appreciated that, in previous studies, anti-tumor antibodies labeled with isotopes have been used successfully to destroy cells in solid tumors animal models, and in some cases in humans. The radionuclides act by producing ionizing radiation which causes multiple strand breaks in nuclear DNA, leading to cell death. The isotopes used to produce therapeutic conjugates typically produce high energy a-, y- or (3-particles which have a therapeutically effective path length. Such radionuclides kill cells to which they are in close proximity, for example neoplastic cells to which the conjugate has attached or has entered. They generally have little or no effect on non-localized cells. Radionuclides are essentially non-immunogenic.

With respect to the use of radiolabeled conjugates in conjunction with the present invention, the antibodies may be directly labeled (such as through iodination) or may be labeled indirectly through the use of a chelating agent. As used herein, the phrases “indirect labeling” and “indirect labeling approach” both mean that a chelating agent is covalently attached to an antibody and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ′″In and <BR> <BR> <BR> <BR> Y.<BR> soy As used herein, the phrases “direct labeling” and “direct labeling approach” both mean that a radionuclide is covalently attached directly to a dimeric antibody (typically via an amino acid residue). More specifically, these linking technologies include random labeling and site-directed labeling. In the latter case, the labeling is directed at specific sites on the antibody, such as the N-linked sugar residues present only on the Fc portion of the conjugates. Further, various direct labeling techniques and protocols are compatible with the instant invention. For example, Technetium-99m labelled antibodies maybe prepared by ligand exchange processes, by reducing pertechnate (Tc04−) with stannous ion solution, chelating the reduced technetium onto a Sephadex column and applying the antibodies to this column, or by batch labelling techniques, e.g. by incubating pertechnate, a reducing agent such as SnC12, a buffer solution such as a sodium-potassium phthalate-solution, and the antibodies. In any event, preferred radionuclides for directly labeling antibodies are well known in the art and a particularly preferred radionuclide for direct labeling is 131I covalently attached via tyrosine residues. Modified antibodies according to the invention may be derived, for example, with radioactive sodium or potassium iodide and a chemical oxidising agent, such as sodium hypochlorite, chloramine T or the like, or an enzymatic oxidising agent, such as lactoperoxidase, glucose oxidase and glucose. However, for the purposes of the present invention, the indirect labeling approach is particularly preferred.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein in their entirety. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation in co-pending application Ser. Nos. 08/475,813,08/475,815 and 08/478,967, incorporated by reference in their entirety herein, are preferably selected to provide high affinity for trivalent metals, exhibit increased tumor-to-non-tumor ratios and decreased bone uptake as well as greater in vivo retention of radionuclide at target sites, i.e., ovarian tumor sites. However, other bifunctional chelators that may or may not possess all of these characteristics are known in the art and may also be beneficial in tumor therapy.

It will also be appreciated that, in accordance with the teachings herein, antibodies may be conjugated to different radiolabels for diagnostic and therapeutic purposes. To this end the aforementioned co-pending applications, herein incorporated by reference in their entirety, disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ′″In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. lIn is particularly preferred as a diagnostic radionuclide because between about 1 to about 10 mCi can be safely administered without detectable toxicity; and the imaging data is generally predictive of subsequent 90Y-labeled antibody distribution. Most imaging studies utilize 5 mCi . . . In-labeled antibody, because this dose is both safe and has increased imaging efficiency compared with lower doses, with optimal imaging occurring at three to six days after antibody administration.

See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the art are credited with the ability to readily determine which radionuclide is most appropriate under various circumstances. For example, 31I is a well known radionuclide used for targeted immunotherapy. However, the clinical usefulness of 131I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as tIn and 90Y. 90Y provides several benefits for utilization in radioimmunotherapeutic applications: the 64 hour half-life of 90Y is long enough to allow antibody accumulation by tumor and, unlike e.g., 131I, 90Y is a pure beta emitter of high energy with no accompanying gamma irradiation in its decay, with a range in tissue of 100 to 1,000 cell diameters.

Furthermore, the minimal amount of penetrating radiation allows for outpatient administration of 90Y-labeled antibodies. Additionally, internalization of labeled antibody is not required for cell killing, and the local emission of ionizing radiation should be lethal for adjacent tumor cells lacking the target antigen.

Effective single treatment dosages (i.e., therapeutically effective amounts) of 90Y-labeled modified antibodies range from between about 5 and about 75 mCi, more preferably between about 10 and about 40 mCi. Effective single treatment non-marrow ablative dosages of 131I-labeled antibodies range from between about 5 and about 70 mCi, more preferably between about 5 and about 40 mCi. Effective single treatment ablative dosages (i.e., may require autologous bone marrow transplantation) of 13′I-labeled antibodies range from between about 30 and about 600 mCi, more preferably between about 50 and less than about 500 mCi. In conjunction with a chimeric antibody, owing to the longer circulating half life vis-a-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ′″In label, are typically less than about 5 mCi.

While a great deal of clinical experience has been gained with 131I and 90Y, other radiolabels are known in the art and have been used for similar purposes. Still other radioisotopes are used for imaging. For example, additional radioisotopes which are compatible with the scope of the instant invention include, but are not limited to, 123I, 125I, 32p, 57COs 64CU, 67Cu, 77Br, 81Rb, 81Kr, 87Sr, 113In, 127Cs, 129Cs, 132I, 197Hg, 203Pb, 206Bi, 177Lu, 186Re, 212Pb, 212Bi, 47Sc, 105Rh, 109Pd, 153Sm, 188Re, 199Au, 225Ac, 211 At, and 213Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include 125I, ′23I, 99Tc, 43K, 52Fe, 467Ga, 68Ga, as well as ′In. Antibodies have also been labeled with a variety of radionuclides for potential use in targeted immunotherapy Peirersz et al. Immunol. Cell Biol. 65: 111-125 (1987). These radionuclides include 88Re and 86Re as well as ′99Au and 67Cu to a lesser extent. U.S. Pat. No. 5,460,785 provides additional data regarding such radioisotopes and is incorporated herein by reference.

In addition to radionuclides, the antibodies of the present invention may be conjugated to, or associated with, any one of a number of biological response modifiers, pharmaceutical agents, toxins or immunologically active ligands. Those skilled in the art will appreciate that these non-radioactive conjugates may be assembled using a variety of techniques depending on the selected cytotoxin. For example, conjugates with biotin are prepared e.g. by reacting the antibodies with an activated ester of biotin such as the biotin N-hydroxysuccinimide ester.

Similarly, conjugates with a fluorescent marker may be prepared in the presence of a coupling agent, e.g. those listed above, or by reaction with an isothiocyanate, preferably fluorescein-isothiocyanate. Conjugates of the antibodies of the invention with cytostatic/cytotoxic substances and metal chelates are prepared in an analogous manner.

Preferred agents for use in the present invention are cytotoxic drugs, particularly those which are used for cancer therapy. Such drugs include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. As noted previously, one skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins.

These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, (3-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above. Among other cytotoxins, it will be appreciated that antibodies can also be associated with a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the antibodies of the present invention comprise cytokines such as lymphokines and interferons. In view of the instant disclosure it is submitted that one skilled in the art could readily form such constructs using conventional techniques.

Another class of compatible cytotoxins that may be used in conjunction with the disclosed antibodies are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked modified antibodies would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

Preferred embodiments of the invention comprise the administration of an anti-ovarian antibody preferably one having ADCC activity, in combination or conjunction with one or more other therapies such, in particular chemotherapy or radiotherapy (i.e. a combined therapeutic regimen). As used herein, the administration of antibodies in conjunction or combination with an adjunct therapy means the sequential, simultaneous, coextensive, concurrent, concomitant or contemporaneous administration or application of the therapy and the subject antibodies. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated antibodies could be administered intravenously followed by tumor localized external beam radiation. In yet other embodiments, the antibody may be administered concurrently with one or more selected chemotherapeutic agents in a single office visit. A skilled artisan (e.g. an experienced oncologist) would be readily able to discern effective combined therapeutic regimens without undue experimentation based on the selected adjunct therapy and the teachings of the instant specification.

In this regard it will be appreciated that the combination of the subject anti-ovarian antigen antibody (with or without cytotoxin) and a chemotherapeutic agent may be administered in any order and within any time frame that provides a therapeutic benefit to the patient. That is, the chemotherapeutic agent and antibody may be administered in any order or concurrently. In selected embodiments the antibodies of the present invention will be administered to patients that have previously undergone chemotherapy. In yet other embodiments, the antibodies and the chemotherapeutic treatment will be administered substantially simultaneously or concurrently. For example, a an ovarian cancer patient may be given the subject antibody while undergoing a course of chemotherapy. In preferred embodiments the modified antibody will be administered within 1 year of any chemotherapeutic agent or treatment. In other preferred embodiments the subject anti-ovarian antibody will be administered within 10, 8, 6, 4, or 2 months of any chemotherapeutic agent or treatment. In still other preferred embodiments the dimeric antibody will be administered within 4, 3, 2 or 1 week of any chemotherapeutic agent or treatment. In yet other embodiments the dimeric antibody will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

It will further be appreciated that the ovarian antigen antibodies used in the instant invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow B reserves. In other preferred embodiments the radiolabeled immunoconjugates disclosed herein may be effectively used with radiosensitizers that increase the susceptibility of the neoplastic cells to radionuclides. For example, radiosensitizing compounds may be administered after the radiolabeled modified antibody has been largely cleared from the bloodstream but still remains at therapeutically effective levels at the site of the tumor or tumors.

With respect to these aspects of the invention, exemplary chemotherapic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRFNCIPLES OF INTERNAL MEDICINE 1774-1788<BR> <BR> <BR> (Kurt J. Isselbacher et al., eds., 13 in ed. 1994) and V. T. DeVita et al., (1997) and the references cited therein for standard dosing and scheduling. These therapies can be used unchanged, or altered as needed for a particular patient, in combination with one or more anti-ovarian antigen antibodies as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide.

Other compatible chemotherapeutic agents include, but are not limited to, 2-chlorodeoxyadenosine (2-CDA), 2′-deoxycoformycin and fludarabine.

The amount of chemotherapeutic agent to be used in combination with the antibodies of the instant invention may vary by subject or may be administered according to what is known in the art. See for example, Bruce A Chabner et al., Antineoplastic Agents, in GOODMAN & GILMAN'S THE PHARMACOLOGICALBASIS OF THERAPEUTICS 1233-1287 ((Joel G. Hardman et al., eds., 9″ed. 1996).

As previously discussed, the antibodies of the present invention, immunoreactive fragments or recombinants thereof are administered in a pharmaceutically effective amount for the in vivo treatment of ovarian cancers or another cancer characterized by overexpression of the antigen. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of the dimeric antibody, immunoreactive fragment or recombinant thereof, conjugated or unconjugated to a therapeutic agent, shall be held to mean an amount sufficient to achieve effective binding with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the antibody.

More specifically, the subject therapies should be useful for reducing tumor size, inhibiting tumor growth and/or prolonging the survival time of tumor-bearing animals.

Accordingly, this invention also relates to a method of treating tumors in a human or other animal by administering to such human or animal an effective, non-toxic amount of antibody. One skilled in the art would be able, by routine experimentation, to determine what an effective, non-toxic amount of antibody would be for the purpose of treating ovarian malignancies. For example, a therapeutically active amount of a antibody may vary according to factors such as the disease stage (e.g., stage I versus stage IV), age, sex, medical complications (e.g., immunosuppressed conditions or diseases) and weight of the subject, and the ability of the antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

For purpose of clarification, “mammal” refers to any animal classified as a mammal, including humans, domestic and farm animals, and zoo, sports, or pet animals, such as dogs, horses, cats, cows, etc. Preferably, the mammal is human.

“Treatment” refers to both therapeutic treatment and prophylactic or preventative measures.

Those in need of treatment of a B cell malignancy e.g., B cell lymphoma, include those already with the disease or disorder as well as those in which the disease or disorder is to be prevented. Hence, the mammal may have been diagnosed as having the disease or disorder or may be predisposed or susceptible to the disease.

In keeping with the scope of the present disclosure, the antibodies of the invention may be administered to a human or other animal in accordance with the aforementioned methods of treatment in an amount sufficient to produce such effect to a therapeutic or prophylactic degree.

The antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of dimeric antibodies according to the present invention may prove to be particularly effective.

Methods of preparing and administering conjugates of the antibody, immunoreactive fragments or recombinants thereof, and a therapeutic agent are well known to or readily determined by those skilled in the art. The route of administration of the antibody or antibodies (or fragment thereof) of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a preferred administration form would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumine), etc. However, in other methods compatible with the teachings herein, the antibodies can be delivered directly to the site of the adverse cellular population thereby increasing the exposure of the diseased antigen positive tissue to the therapeutic agent.

Preparations for parenteral administration includes sterile aqueous or non-tumor aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof.

The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a dimeric antibody by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations maybe packaged and sold in the form of a kit such as those described in co-pending U.S. Ser. No. 09/259,337 and U.S. Ser. No. 09/259,338 each of which is incorporated herein by reference.

Such articles of manufacture will preferably have labels or package inserts indicating that the associated compositions are useful for treating a subject suffering from, or predisposed to B cell neoplastic disorders.

The availability of isolated protein allows for the identification of small molecules and low molecular weight compounds that inhibit the binding of protein to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbance, fluorescence or luminescence as read-outs.

[Gonzalez, J. E. et al., Curr. Opin. Biotech. 9: 624-631 (1998)].

Model systems are available that can be adapted for use in high throughput screening for compounds that inhibit the interaction of a protein with its ligand, for example by competing with the protein for ligand binding. Sarubbi et al., Anal. Biochem. 237: 70-75 (1996) describe cell-free, non-isotopic assays for discovering molecules that compete with natural ligands for binding to the active site of IL-1 receptor. Martens, C. et al., Anal. Biochem. 273 20-31 (1999) describe a generic particle-based nonradioactive method in which a labeled ligand binds to its receptor immobilized on a particle; label on the particle decreases in the presence of a molecule that competes with the labeled ligand for receptor binding.

The polynucleotides and polypeptides of the present invention may be utilized in gene delivery vehicles. The gene delivery vehicle may be of viral or non-viral origin (see generally, Jolly, Cancer Gene Therapy 1: 51-64 (1994); Kimura, Human Gene Therapy 5: 845-852 (1994); Connelly, Human Gene Therapy 1: 185-193 (1995); and Kaplitt, Nature Genetics 6: 148-153 (1994)). Gene therapy vehicles for delivery of constructs including a coding sequence of a therapeutic according to the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, CancerRes. 53: 3860-3864 (1993); Vile and Hart, Cancer Res. 53: 962-967 (1993); Ram et al., Cancer Res. 53: 83-88 (1993); Takamiya et al., J. Neurosci. Res. 33: 493-503 (1992); Baba et al., J. Neurosurg 79: 729-735 (1993); U.S. Pat. No. 4,777,127; GB Patent No. 2,200,651; and EP 0 345 242.

Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63: 3822-3828 (1989); Mendelson et al., Virol. 166: 154-165 (1988); and Flotte et al., P. N. A. S. 90: 10613-10617 (1993).

Representative examples of adenoviral vectors include those described by Berkner, Biotechniques 6: 616-627 (Biotechniques); Rosenfeld et al., Science ˜252: 431-434 (1991); WO 93/19191; Kolls et al., P. N. A. S. 215-219 (1994); Kass-Bisler et al., P. N. A. S. 90: 11498-11502 (1993); Guzman et al., Circulation 88: 2838-2848 (1993); Guzman et al., Cir. Res. 73: 1202-1207 (1993); Zabner et al., Cell 75: 207-216 (1993); L1 et al., Hum. Gene Ther. 4: 403-409 (1993); Cailaud et al., Eur. J. Neurosci. 5: 1287-1291 (1993); Vincent et al., Nat. Genet. 5: 130-134 (1993); Jaffe et al., Nat. Genet. 1: 372-378 (1992); and Levrero et al., Gene 101: 195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3: 147-154 (1992) may be employed.

Other gene delivery vehicles and methods may be employed, including polycationic condensed DNA linked or unlinked to killed adenovirus alone, for example Curiel, Hum. Gene Ther. 3: 147-154 (1992); ligand-linked DNA, for example see Wu, J. Biol. Chem. 264: 16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14: 2411-2418 (1994), and in Woffendin, Proc. Natl. Acad. Sci. 91: 1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13 796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91 (24): 11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

### EXAMPLES

While the invention has been described supra, including preferred embodiments, the following examples are provided to further illustrate the invention.

### Example 1

Gene Identification Table 1 summarizes the information for the gene sequences overexpressed by ovarian tissues that were identified using the Gene Logic GeneExpress Oncology DataSuite. The column titled ‘Gene Logic EST’ contains the Genbank accession numbers for the ESTs identified as overexpressed in ovarian tumors compared to normal tissue in the GeneExpress database.

These ESTs were then queried in the UniGene database (a public database that is part of NCBI-www.ncbi.nlm.nih.gov/UniGene) to identify longer ESTs corresponding to the same gene. The Genbank accession numbers for these ESTs are listed in the next column under the heading ‘Representative EST’. These representative ESTs were ordered from the American Type Culture Collection (ATCC) and catalog numbers for each are listed in the third column. If information on the chromosomal location of the gene sequences was available in the NCBI database it is listed in the next column.

All of these DNA sequences were translated into protein sequence if possible, and the predicted protein sequences were analyzed using two internet based algorithms designed to predict transmembrane domains in proteins. This information is listed in the column titled ‘Predicted TM.’ The abbreviation ‘NT’ means the DNA sequence was not translatable, therefore the analysis could not be performed. Proteins containing transmembrane domains are more likely to be expressed on the cell surface, making them suitable targets for antibody therapy.

Identification of such proteins is therefore highly desired.

### Example 2

Gene Expression Table 2 summarizes the actual expression levels for each of the candidate ESTs, as measured using the Gene Logic GeneExpress OncologyDatasuite. This comparison was made by creating a data set containing all ovarian tumor samples and comparing this to a data set containing all normal ovary, kidney, liver, lung, colon, pancreas and breast samples. The overall fold increase in expression levels in the ovary tumor data set compared to the normal data set is shown in column 2. The median expression level measured for each tissue type is reported in the next column, underneath which is the range of expression measured in that tissue type. The total number of samples for each tissue type is as follows: ovary tumor, 13; normal ovary, 20; normal kidney, 19; normal liver, 20; normal lung, 21; normal colon, 25; normal pancreas, 10; normal breast, 19. The expression values were obtained directly from the Oncology DataSuite and were determined using Gene Logic's proprietary normalization algorithm. An entry of zero indicates that none of the samples of the corresponding tissue type had detectable expression of the EST. A median entry with no range indicates expression was detectable in a single sample of the corresponding tissue type.

The data in Table 3 is a continuation of that presented in Table 2, this time showing the percentage of each tissue type expressing the indicated EST. The total number of samples for each tissue type is described above.

Table 1: Identification of Candidate DNA Sequences Overexpressed in Ovarian Tumors using the Gene Logic GeneExpressT Oncology DataSuite Gene Logic EST Representative ATCC Chromosome Predicted TM EST Catalog # Domains AI683094 AI683094 3460054 22q12. 1 NT AI821669 AI866319 3623437 8qll None AI498957 W84863 872889 19pu3. 3 NT AI923224 AI537678 3387915 19pu3. 3 4 AI092936 AI801043 3383209 unclear 7-10 AI742002 AI868025 3098351 19pu3. 3 1 AI219073 AI688913 3462070 19q13. 4 1 AI741736 AI539017 3396839 unclear 1 AI871120 AI871120 3628201 10q21. 2 None AI924459 AA830718 1620833 3q13. 2 None Table 2: Expression Data for Candidate ESTs as Determined Using the Gene Logic GeneExpress™ Oncology DataSuite<BR> Expression Intensity, Gene Logic Units (Median and Range) Gene Logic Fold Increase Normal Tissues EST Tumor v. Mixed Ovary Tumor Ovary Kidney Liver Lung Colon Pancreas Breast Normals AI683094 11 376 558 0 82 0 0 0 0 135-2905 AI821669 7.5 245 204 0 16 51 0 0 42 106-816 69-339 34-84 11-69 A1498957 6 693 0 0 0 0 0 0 0 447-1102 AI923224 19 2286 302 0 0 97 0 0 108 106-3704 95-508 58-116 68-147 AI092936 5 536 0 68 234 111 257 150 95 262-640 1-129 63-358 1-412 76-114 AI742002 6.4 690 270 327 214 211 177 0 152 181-3260 104-738 297-351 58-1511 101-1036 108-533 AI219073 10 781 542 261 452 129 498 0 162 79-2165 186-315 51-1080 248-836 9-387 AI741736 10 556 259 61 191 0 233 61 47 227-1136 199-321 38-126 30-117 AI871120 5 1411 0 0 0 0 0 0 0 963-2183 AI924459 8 260 119 0 0 263 0 0 0 103-2908 35-469 Table 3: Percentage of Tissue Samples Expressing the Candidate ESTs<BR> Normal Tissues<BR> Gene Logic<BR> ESt Ovary Tumor Ovary Kidney Liver Lung Colon Pancreas Breast<BR> AI683094 77% 5% 0 5% 0 0 0 0<BR> AI821669 85% 10% 0 5% 43% 0 0 26%<BR> AI498957 38% 0 0 0 0 0 0 0<BR> A1923224 92% 10% 0 0 19% 0 0 42%<BR> AI092936 54% 0 21% 5% 19% 40% 10% 15%<BR> AI742002 69% 20% 16% 5% 33% 28% 0 37%<BR> AI219073 85% 5% 32% 5% 38% 16% 0 68%<BR> AI741736 77% 10% 63% 5% 0 4% 10% 16%<BR> AI871120 31% 0 0 0 0 0 0 0<BR> AI924459 77% 5% 0 0 76% 0 0 0 Example 3 Nucleotide and Amino Acid Sequences The nucleotide sequence of each candidate EST is detailed in this section. These sequences are obtained directly from the Genbank entries in the public NCBI database (www.ncbi.nlm.nih.gov.) Nucleotide sequence for both the Gene Logic and Representative ESTs for each candidate are listed, with homologous sequence shown in bold for each EST. Additional sequence information obtained for two of the candidates is reported where indicated.

1. Gene Logic EST AI683094 There is no representative EST for this sequence. Additional sequence information obtained by sequencing the ATCC clone containing this EST is shown below. The underlined sequence is the reverse complement of EST AI683094.

2. Gene Logic EST AI821669 Representative EST AI866319 There is no overlap between these two ESTs. The Gene Logic EST AI821669 comprises the 3′ end of IMAGE clone 740416. The 5′ end of this IMAGE clone is AI820919, which is the reverse complement of the representative EST AI866319 above.

Additional sequence information obtained by sequencing the ATCC clone containing representative EST AI866319 is shown below. The underlined region is the reverse complement of the EST.

This additional sequence was searched against the Genbank nr database and found to match portions of Accession number NM_{—}011441, shown below. This sequence is identified as the mouse gene Sox17. This gene is described in the following publication; Kanai, Y., Kanai-Azuma, M., Noce, T., Saido, T. C., Shiroishi, T., Hayashi, Y. and Yazaki, K. (1996) Identification of Two Sox17 Messenger RNA Isoforms, With and Without Differential Expression in Mouse Spermatogenesis. J. Cell Biol., 133 (3), 667-681. Regions of sequence similarity between the above sequence and the Sox17 sequenced are shown in bold. Based on this sequence similarity, it appears that the gene identified initially as Gene Logic EST AI821669 represents the human homolog of the mouse Sox17 gene.

Genbank Accession &num;NM_{—}011441 (Sox17) 3. Gene Logic EST A1498957 Representative EST W84863 The sequences shown in bold in the above ESTs are the reverse complement of each other.

4. Gene Logic EST AI923224 Representative EST AI537678 The sequences in bold in the above ESTs are homologous. The representative EST AI537678 was found to match accession number AK024365 in the Genbank nr database, the sequence of which is listed below. This Genbank entry is defined as ‘homo sapiens cDNA FLJ14303fis, clone PLACE2000132’, and was a direct submission from the NEDO cDNA sequencing project (Helix Research Institute, Kisarazu, Chiba, Japan. T. Isogai, T. Otsuki, authors.) The underlined sequence in the EST above is the reverse complement of the underlined sequence in Genbank accession # AK024365 shown below.

Genbank Accession #AK024365 5. Gene Logic EST AI092936 Representative EST AI801043 (SEQ ID NO: 13) The representative EST AI801043 was found to match accession number NM024531 in the Genbank nr database, the sequence for which is listed below. This Genbank entry is defined as ‘homo sapiens hypothetical protein FLJ 11856’ and was a direct submission by Robert Strausberg at CGAP (Cancer Genome Anatomy Project. Public domain-http://cgap.nci.nih.gov.) The reverse complement of EST AI801043 in its entirety corresponds to the portion of NM024531 underlined below.

Genbank Accession #NM_{—}024531 6. Gene Logic EST AI742002 (SEQ ID NO: 15) Representative EST AI868025 7. Gene Logic EST AI219073 (SEQ ID NO: 17) Representative EST A1688913 The Gene Logic EST AI219073 was found to match to a portion of accession # AF282167 in the Genbank nr database, the sequence of which is shown below. This sequence is defined as ‘homo sapiens DRC3 mRNA’, and was a direct submission from the National Laboratory of Molecular Oncology Cancer Institute, Panjiayuan, Chaoyang Qu, Beijing, China.

The sequence is also described in the following publication; Wu, K., Xu, Z., Wang, M., Xu, X., Han, Y., Cao, Y., Wang, R., Sun, Y. and Wu, M. (1999.) Cloning and Expression Analyses of Down Regulated cDNA C6-2A in Human Esophageal Cancer. Chung-Hua I Hsuch I Chuan Hsuch Tsa Chih, 16 (5), 325-327. The reverse complement of Gene Logic EST A1219073 in its entirety corresponds to the underlined sequence in AF 282167 below.

Genbank Accession # AF282167 8. Gene Logic EST AI741736 Representative EST AI539017 The protein sequence below is the translation product of Genbank accession number AB037805, ‘homo sapiens mRNA for KIAA1384’, which corresponds to Gene Logic EST AI741736. The protein contains a predicted transmembrane domain, which is underlined.

The Gene Logic EST AI74 1736 was found to match a portion of accession number AB037805 in the Genbank nr database, the sequence of which is shown below. This sequence is defined as ‘homo sapiens mRNA for KIAA1384 protein’ and was a direct submission by the Kazusa DNA Research Institute, Kisarazu, Chiba, Japan. The sequence may be described in the following article; Nagase, T. et al (2000.) Prediction of the Coding Sequences of Unidentified Human Genes. XVI. The Complete Sequences of 150 new cDNA Clones from Brain Which Code for Large Proteins in vitro. DNA Res., 7 (1), 65-73. The underlined sequence in the Gene Logic EST is the reverse complement of the underlined sequence contained within AB037805 below. The protein encoded by AI741736 is provided (SEQ ID NO: 23) based on the transmembrane region, which appears to be expressed on the surface of ovarian cells.

Genbank Accession # AB037805 9. Gene Logic EST AI871120 There is no representative EST for this sequence.

10. Gene Logic EST AI924459 Representative EST AA830718 Example 4 Identification of Anat 2 This example describes the characterization of a novel gene, herein named “Anat 2”, a fragment of which was identified using the Gene Logic Gene Express Oncology Datasuite.

The gene fragment, an EST with Genbank accession number AA977 181, was identified in a Datasuite search comparing gene expression in ovarian papillary serous adenocarcinomas with expression in normal tissues.

FIG. 1 is an ‘electronic Northern’ depicting the gene expression profile of this fragment as determined using the Gene Logic datasuite. The figure shows that the total number of samples for each tissue type is as follows: ovary tumor, tumor % above 50, 35; ovary tumors update, 46; normal breast, 35; normal colon, 28; normal esophagus, 18, normal kidney, 25; normal liver, 21; normal lung, 32; normal lymph node 10; normal ovary, 25; normal pancreas, 17; normal prostate, 15; normal stomach, 25.

Ovary tumor, tumor % above 50′ refers to tumor samples for which at least 50% of each sample comprises malignant tissue, as determined by a pathologist. This sample set is a subset of ‘ovary tumors update’, which comprises all ovary tumor samples contained within the Gene Logic database.

An additional 3 genes with significant homology to Anat 2 were identified by searching the NCBI human genome databases (public domain information, available through www.ncbi.nlm.nih.gov.) These homologous genes have therefore been named the Anat family.

Table 4 below summarizes the information available on the Anat genes from the NCBI databases.

Table 4: The Anat Family NCBI Family Name Chromosome Comments Gene Name Location KIAA0416 Anat 1 5q31. 2 contained in intron of cateninpl gene FLJ32082 Anat 2 2 p12 contained in intron of catenin β 2 gene FLJ12568 Anat 3 2 p12 Anat 4 1Oq22 homolog of macaque brain hypothetical protein.

At least 1 mouse Anat homolog exists in addition to the above four human Anat genes, which suggests that the Anat gene family is conserved across different species.

Provided below are the nucleotide sequences of all four human Anat genes. The Genbank accession number for each of the sequences is also provided as a reference.

Anat 1/KIAA0416/Genbank Accession # AB007876 Anat 2/Gene Logic Candidate AA977181/FLJ32082/Genbank Accession # AK056644 Anat 3/extended FLJ12568/Genbank Accession # NM024993 Anat 4/human homologue of macaca hypothetical protein/Genbank Accession # AB060846 Shown below are the translated protein sequences of each of the Anat genes.

Anat 1/KIAA0416/Genbank Accession # BAA24846 Anat 2/FLJ32082/Genbank Accession # BAB71240 Anat 3/extended FLJ12568/Genbank Accession &num; NP079269 Anat 4/human homologue of macaca hypothetical protein/Genbank Accession # BAB46868 Sequence analysis using internet based proteomics programs predict each of the Anat proteins to be type I transmembrane proteins containing leucine rich repeat regions on their extracellular domains. All four Anat proteins share a high degree of homology, as illustrated in Table 5 below.

Table 5: Comparison of protein similarities between Anat family members.

The numbers in bold indicate % amino acid identity; numbers in parentheses indicate % amino acid similarity.

As the Gene Logic expression profile for Anat 2 (FIG. 1) indicates this gene is overexpressed in ovarian tumors, additional research has been undertaken to further characterize this gene. Several EST clones corresponding to portions of the Anat 2 gene were ordered from the American Type Culture Collection (ATCC, Manassas, Va.) and sequenced to confirm their identity as Anat 2. The EST clones are listed in Table 6 below.

Table 6: Anat 2 EST clones obtained by IDEC GenBank Accession # IMAGE clone # ATCC catalogue # AW161290 (5′) 2782579 5006089 AW157718 (3′) BE551640 3195647 5421514 AW874138 3126137 5249423 AA977181 1587374 3209174 The expression of Anat 2 in normal human tissues was further investigated by PCR experiments using commercially available human cDNA panels and cDNA samples prepared in-house from human tissues and cell lines. The results of these experiments are presented below in FIG. 2. The following PCR primers were synthesised and used in the experiments in panels a, b, c and d below: The sequence of these primers is contained in the portion of Anat present in IMAGE clone # 3126137, plasmid DNA from which was used as a positive control in each experiment.

A PCR product of 442 bp is obtained from any cDNA template containing the Anat gene.

FIG. 2(a) shows the expression of Anat-2 in normal tissues, as determined using Clontech's human normal multiple tissue cDNA panel (MTC panel, catalog # K1421-1) Upper panel; Anat expression, lower panel; Glyceraldehyde 3-phosphate dehydrogenase (GAPDH) expression. GAPDH is a housekeeping gene expressed at high levels in all human tissues and is used here as a control for cDNA integrity. The cDNA samples present in each lane are indicated on the figure. The positive control is plasmid DNA for IMAGE clone 3126137; the negative control is water (no template.) The data in this panel indicates that Anat-2 is expressed weakly in heart, brain, liver and small intestine, and is absent from all other normal tissues.

Anat-2 expression in normal heart and brain was investigated further due to the results seen in FIG. 2(a). Expression in normal heart was next examined using Clontech's human cardiovascular multiple tissue cDNA panel (catalog # K1427-1.) The results of this experiment are shown in FIG. 2(b.) Each heart sample represents a pool of multiple donors (3-39.) The upper panel depicts Anat-2 expression; the lower panel depicts GAPDH expression. The results of this experiment indicate that Anat-2 is not expressed in any heart tissue. As the data in panels (a) and (b) appear contradictory, it is somewhat ambiguous as to whether Anat 2 is truly detectable in human heart.

FIG. 2(c) depicts Anat-2 expression in brain tissue using human brain cDNA panels from Biochain Institute (catalog &num;s 0516011 and 0516012.) Brain sections in each sample are indicated on the figure. The upper panel shows Anat-2 expression, the lower panel shows GAPDH expression. The data in this figure corroborates that seen in FIG. 2(a), and indicates that Anat-2 is expressed weakly in several brain compartments. As the samples used in this panel represent individual donors and not pooled material, this experiment should not be seen as definitive, and further investigation of Anat-2 brain expression is warranted.

FIG. 2(d) depicts Anat-2 expression in a panel of human ovarian tumor samples and 2 ovarian tumor cell lines. The ovarian tumor samples were obtained from the Cooperative Human Tissue Network (CHTN); the cell lines Ovcar-3 and PA1 were obtained from the ATCC. RNA was isolated from each sample and cell line using Qiagen's RNeasy kit (catalog &num; 75162). cDNA was prepared from total RNA using Gibco BRL cDNA synthesis system (Life Technologies, catalog # 18267-021.) The upper panel shows Anat-2 expression, the lower panel shows GAPDH expression in FIG. 2(d). The numbers above each lane correspond to ovarian tumor samples as follows: 6044: moderately differentiated cystadenocarcinoma 7791: poorly differentiated papillary serous adenocarcinoma 7333 poorly differentiated papillary serous adenocarcinoma 7291: poorly differentiated endometriod adenocarcinoma 6841: papillary serous adenocarcinoma 7070: endometriod adenocarcinoma 7120: poorly differentiated adenocarcinoma 7723: poorly differentiated papillary serous adenocarcinoma The data in this panel indicates that Anat-2 is expressed strongly in four of the tumor samples, and weakly in an additional three samples. It is also expressed in both of the ovarian tumor cell lines.

### Example 5

Cloning and Expression Analysis of Anat-2 Full length Anat-2 open reading frame was assembled by PCR from IMAGE clones 2782579 and 1587374 (obtained from ATCC, Rockville, Md.) Full length open reading frames for Anats 1, 3 and 4 were cloned from chromosomal DNA obtained from Jurkat cells (human T-cell line, ATCC, Rockville, Md.) using standard molecular techniques. The following sections describe expression of numerous Anat constructs. In all cases, Anat genes were cloned into IDEC's proprietary mammalian expression vectors containing a C-terminal tag. All experiments use a human B7 construct (either B7.1 or B7.2) in parallel with the Anat constructs as a positive control. These are related and well characterized cell surface proteins used for control purposes.

Determination of Anat-2 Cell Surface Expression in Transiently Transfected COS cells.

COS7 cells were transiently transfected with Anat-2 or control expression vectors (3.5 micrograms of DNA per 100 mm tissue culture dish of cells) using LipofectAMINE reagent (Invitrogen) according to the manufacturer's instructions. Cell surface expression was analyzed 48 hrs post-transfection by employing the EZ-Link Sulfo-NHS-LC-Biotin Kit (Pierce Chemical Co.) in conjunction with a modified version of the protocol described by Altin et al. (1995) Anal. Biochem., 224: 382-389. Briefly, triplicate samples of transfected cells were washed four times with ice-cold PBS (pH 8) (Irvine Scientific), then incubated at room temperature with 2.5 ml of 0.54 mM Sulfo-NHS-LC-Biotin (dissolved in PBS) per 100 mm dish. Subsequently, the cells were washed four times with ice-cold PBS, and lysed in 0.5 ml of RIPA buffer (Upstate Biotech.). Insoluble material was removed by centrifugation, and protein concentration of the supernatants was determined using the Micro-BCA kit (Pierce Chemical Co.), according to the manufacturer's instructions. For the isolation of biotinylated proteins, 500 μg of total protein was diluted with RIPA buffer to a total volume of 1.4 ml per sample. The diluted cell lysates were incubated with 100 il of immobilized Strepavidin beads (Pierce Chemical Co.) with gentle mixing for 1 hour at 4° C., followed by extensive washing (8 times) with RIPA buffer. Elution of the biotinylated proteins was achieved by boiling for 5 min in SDS-PAGE sample buffer. The triplicate samples were pooled, separated by SDS-PAGE and analyzed by immunoblotting using a proprietary monoclonal antibody to the C terminal tag.

FIG. 7 shows an immunoblot of total proteins (25 Ag) from the cell lysates (lanes: 1, 3, and 5) or biotinylated proteins isolated on Streptavidin beads (lanes: 2, 4 and 6) Two different preparations of the Anat2 expression vector were used to transfect the cells; lanes 1 and 2 for DNA preparation 1, and lanes 3 and 4 for DNA preparation 2. Lanes 5 and 6 correspond to B7.2.

The positions of the Anat-2 and B7.2 bands are indicated. The increase in molecular weight of the biotinylated Anat-2 in relation to the major Anat-2 band detected in the total cell lysate is likely to reflect glycosylation of the cell surface protein. The detection of biotinylated Anat-2 (lanes 2 and 4) indicates that the protein is present at the cell surface.

Generation of Anat-2 Expressing Stable Chinese Hamster Ovary (CHO) Cell Lines and Determination of Cell Surface Expression. Full length Anat-2 contained in IDEC's proprietary mammalian expression vector was transfected into DHFR-CHO DG44 cells (Urlaub et. al., Som. Cell. Mol. Gen., 12: 555-566, 1985) by electroporation. Briefly, cells were washed, counted and resuspended in ice cold SBS buffer (7 mM NaPO4, 1 mM MgC12, 272 mM sucrose, pH 7.4).

Plasmid DNA was linearized by restriction digestion and 1, 2, or 3 ug/ml DNA mixed with 4×106 DG44 cells and electroporated. Cells were seeded into 96-well microtiter culture plates and cell lines selected for G418 resistance in CHO S SFM II media (Gibco) supplemented with hypoxanthine+thymidine (HT, Gibco). Wells from the plates transfected with the lowest concentration of DNA and exhibiting robust cellular growth were expanded into 24 well plates and then T25 cell culture flasks for analysis. Cell lines were screened for expression of Anat-2 by Western Blot analysis. Briefly, cells were collected from confluent T25 culture flasks by centrifugation, counted and washed with PBS pH 7.4. and lysed at a concentration of 500 ul of RIPA buffer/3×106 cells (RIPA buffer=50 mM Tris-HCl pH 7.4, 1% NP-40, 0.25% Na deoxycholate, 150 mM NaCl, 1 mM EDTA, 1 mM PMSF, 1 ug/ml Aprotinin, 1 mM sodium vanadate, and 1 mM sodium fluoride). The concentration of total protein in the cellular lysates was determined by the BCA assay (Pierce) according to the manufacturer's instructions. Lysate concentrations were adjusted to load equivalent amounts of total protein, electrophoresed on a 4-20% Tris-glycine SDS-PAGE gel (Invitrogen) and electrophoretically transferred onto a nitrocellulose membrane (Hybond ECL). Nonspecific sites were blocked with PBS containing 5% nonfat milk (w/v)+0.1% Tween 20, pH7.4 and then probed with a monoclonal antibody against the C-terminal tag. Immune complexes were detected by incubating the membrane with an HRP-conjugated goat anti-mouse IgG and after sufficient washing, developing the membrane with ECL reagent (Amersham). Results of a typical western are shown in FIG. 8. The predicted mobility of Anat-2 is indicated by an arrowhead. This figure shows 8 transfected cell lines (lanes 1-8) along with untransfected CHO (lane 9) as a negative control. This screening method was used to identify the top producing Anat-2 cell line (lane 8 in the figure), which was expanded in culture in 125 ml spinner flasks. This cell line was subsequently used as the positive control in screening for Anat-2 specific monoclonal antibodies (see section entitled ‘Generation of Anti-Anat-2 Murine Monoclonal Antibodies’ below.) Surface expression of Anat-2 in the above described stable CHO cell lines was determined using the EZ-Link Sulfo-NHS-LC-Biotin Kit (Pierce Chemical Co.) The methodology was essentially as described in FIG. 3 for biotinylation of transfected Cos cells.

Biotinylated proteins were isolated from whole cell lysates using immobilized Streptavidin and subjected to SDS-PAGE and immunoblotting using a proprietary monoclonal antibody to the C terminal tag. FIG. 9 shows an immunoblot for eight different stable CHO cell lines expressing Anat2 (lanes 1-8), and one expressing B7.2 (lane 9) as a positive control. The positions of Anat-2 and B7.2 bands, and the molecular weight markers (in kDa) are indicated. The presence of biotinylated Anat-2 in six of the cell lines (lanes 2-7) indicates that the protein is present at the cell surface.

An Anat-2 Ig immunoadhesin consisting of the extracellular domain of Anat-2 genetically fused to a human IgG1 Fc domain was constructed in order to generate a soluble form of the Anat-2 protein. The extracellular portion of Anat-2 was generated as a BglII-NheI DNA fragment by PCR methodology from the full length Anat-2 template. The fragment was inserted into the Bgl II and NheI sites of a proprietary mammalian expression vector containing the IgG Fc domain. This resulted in an in-frame fusion of the Anat sequence with the N-terminus of the IgG sequence. The Anat-2 Ig immunoadhesin construct was then transfected into the DHFR-CHO DG44 cell line and cultured as described above for full length Anat-2. Cell lines were screened for secretion of soluble Anat-2 Ig immunoadhesin by ELISA. Briefly, Immulon II plates (Thermo Labsystems) were coated with goat anti-human IgG and nonspecific sites blocked. Supernatants from Anat-2 Ig immunoadhesin G418 resistant cell lines were diluted into binding buffer (0.5% non-fat milk in PBS) and added to the plates. Captured immune complexes were detected by incubating with HRP-conjugated goat anti-human IgG (Southern Biotechnology) and developed with TMB Peroxidase substrate (KPL Inc.) Color development was quenched by the addition of 2N H2SO4, and absorbencies were measured using a microtiter pate reader (Molecular Dynamics) at a dual wavelength setting of 450/540 nm. To identify top producing cell lines the IgG reactivity of supernatants were compared to a B7 IgG immunoadhesin standard. This method was used to determine the top producing cell line, which was then expanded in culture. Anat-2 Ig purified from this culture was subsequently used as immunogen for Anat-2 monoclonal antibody development (see below).

Generation of Anti-Anat-2 Murine Monoclonal Antibodies. Anat-2 Ig protein was purified from the supernatant of Anat-2 Ig expressing CHO cell lines using a protein-A affinity column and used as an immunogen to generate Anat-2 specific monoclonal antibodies. Male Balb/c mice were injected with the purified protein following a proprietary rapid immunization protocol consisting of 5 sets of 12 injections over an 11 day period. Mice were bled on day 12, and the titer of Anat-2 specific antibodies was determined by ELISA on 96 well plates coated with purified Anat-2 Ig protein. On day 13, spleens from mice exhibiting the highest titer were removed and fused to mouse myeloma Sp2/0 cells following standard immunological techniques (Kohler, G. and Milstein, C. 1975. Nature 256, p 495.) The resulting hybridoma cells were plated in 96-well flat bottom plates (Corning) and cultured in Iscove's Modified Dulbecco's Medium (IMDM, Irvine Scientific) containing 10% FBS, 4 mM L-Glutamine (Gibco), 1× non-essential amino acids (Sigma), 1 mM sodium pyruvate (Sigma), 5 ug/ml gentamicin (Gibco) supplemented with HAT (5×10-3 M hypoxanthine, 2×10-5M aminopterin, 8×10-3M thymidine, Sigma) and 1% Origen hybridoma cloning factor (Igen International.) After 5 days in culture, the medium was replaced with IMDM containing the above supplements plus HT (Gibco) in place of HAT.

After 11 days of culture, supernatants were screened for reactivity against Anat-2 Ig protein by ELISA. Briefly, single well supernatants were transferred to Immulon-II plates (Thermo Labsystems) coated with 2 ug/ml of purified Anat-2 Ig fusion protein in bicarbonate buffer.

Positive clones from this assay were then screened against purified B7.1 Ig as a negative control.

Clones showing highest activity against Anat-2 Ig and little or no activity against B7.1 Ig were rescreened in duplicate, and the highest producing clones were selected for subcloning and expansion. Nine clones were ultimately expanded up to 125 ml spinner flasks in ISPRO media (Irvine Scientific) supplemented with 5% low IgG FBS (Hyclone), HT and 1% cloning factor.

Antibodies were purified from culture supernatants by protein-A affinity chromatography after 10-12 days, and isotype determination was performed using a Mouse Immunoglobulin ELISA kit (Pharmingen) according to the manufacturers instructions.

FIGS. 10(a) and 10 (b) depict the reactivities of 7 IgG Kappa anti-Anat2 monoclonal antibodies generated as described above.

FIG. 10(a) shows the results of an ELISA measuring binding of the antibodies to Anat2-Ig compared to B71-Ig. Briefly, serial dilutions of protein-A purified antibodies were incubated in Immulon-II plates coated with either purified Anat-2 Ig or B7.1 Ig at 2 ug/ml in bicarbonate buffer. Anti-B7 (Pharmingen) was used as a positive control for the B7 Ig plate. All dilutions and incubations were carried out in PBS containing 1% non-fat milk and 0.05% Tween-20. After incubation for 1 hour at room temperature, plates were washed 12 times with tap water then incubated with goat anti-mouse IgG HRP (Southern Biotechnology) at 1: 2000 dilution. Plates were incubated for 1 hour at room temperature, washed as described above, then incubated with TMB peroxidase substrate (KPL) until color developed. The enzymatic reaction was quenched by the addition of 4N H2SO4, and absorbance was measured at 450 nm using a Titertek Multiskan MCC/430 plate reader.

The data in FIG. 10(a) clearly shows specificity of binding to Anat2-Ig rather than B7-Ig for all seven antibodies tested, demonstrating that the antibodies are specific for the Anat-2 antigen.

FIG. 10(b) shows the results of a FACS assay measuring binding of 6 of the above Anat-2 antibodies to stably transfected Anat-2 CHO cells. Briefly, Anat-2 CHO stable transfectants and untransfected CHO cells (negative control) were permeabilized by incubation in Dulbecco's phosphate buffered saline (D-PBS) containing 2% FBS, 0.05% NaN3, 10% goat serum and 0.05% saponin. Cell concentrations were adjusted to 2×106/ml, and 50 ul of cell suspensions were incubated with serial dilutions of protein-A purified Anat-2 monoclonal antibodies in 96 well flat bottom plates (Corning.) All dilutions, incubations and washes were carried out using the above described buffer. Plates were incubated for 45 minutes on ice, washed twice, then incubated with goat anti-mouse IgG-RPE secondary antibody diluted 1: 500 (Southern Biotechnology.) Plates were again incubated for 45 minutes on ice, washed twice, then cells were transferred to 12×75 mm tubes and fluorescence intensity was measured using a Beckton Dickinson FACS calibur cytometer. The data in FIG. 10(b) shows specific binding of the Anat-2 antibodies to the Anat-2 CHO transfectants over the untransfected CHO cells, indicative that these antibodies specifically recognize the Anat-2 antigen.

An anti Anat-2 murine monoclonal antibody referred to as 6B8 was selected for further characterization because of its high titer and Anat-2 binding specificity demonstrated in FIG. 10.

Confirmation of Specificity of Anti Anat-2 Murine Monoclonal Antibody 6B8. As all Anat family members share a significant degree of homology, the following experiment was conducted to ensure that 6B8 antibody was specific for Anat-2. Soluble immunoadhesion constructs of Anat family members 1, 2 and 3 were constructed by fusing the extracellular domain of the each Anat to a human IgG1 Fc domain as described earlier. COS7 cells were transiently transfected with empty vector, positive control vector containing human IgG1 control or Anat-Ig fusion vectors (all using 6 ig of DNA/100 mm dish) for 6 hours using Lipofectamine reagent (Invitrogen, 18324-012) according to the manufacturer's instructions. The transfection medium was subsequently removed, and the cells incubated for 18 hrs in complete growth medium (DMEM supplemented with 10% FBS, 0.292 mg/ml L-Glutamine, and 1 mM Sodium pyruvate). The cells were washed one time with PBS, then incubated for a further 36 hrs in serum-free medium (DMEM supplemented with 0.292 mg/ml L-Glutamine, and 1 mM Sodium pyruvate). The transfected cells were lysed in 0.5 ml of 2×SDS gel loading buffer (Invitrogen) and boiled for 5 min. Samples were electrophoresed on a 10% Bis-Tris gel (Invitrogen) and transferred to PVDF membrane. Immunoblotting was performed using Goat Anti-Human IgG-HRP (Southern Biotechnology Associates, Inc) to detect expression of Ig fusion proteins, or anti-Anat-2 murine monoclonal antibody 6B8 (1 pg/ml), followed by goat anti-mouse-HRP antibody (BioRad) secondary antibody (1: 2000). The blots were detected using ECL (Amersham.) FIG. 11 shows the results of this experiment. The mobility of Anat-2 is denoted by an arrowhead. The data in this figure demonstrates that anti Anat-2 monoclonal antibody 6B8 specifically recognizes Anat 2, as no reactivity with the related protein Anat-3 was observed. Anat-1 was not expressed in this experiment.

Ovarian Carcinoma Tissue Staining with Anti Anat-2 Monoclonal Antibody 6B8.

Immunohistochemical data demonstrating surface binding of Anat-2 monoclonal antibody 6B8 to an ovarian carcinoma cell is presented in FIG. 12. Ovarian teratocarcinoma cell line PA-1 (ATCC, Rockville, Md.) plated on glass coverslips were washed twice in 1× phosphate buffered saline (PBS), then fixed for 10 minutes in 3.7% Formaldehyde, 3% Sucrose in 1×PBS at room temperature. Coverslips were then washed three times for 10 minutes each with PBS and incubated for 1 hour at room temperature with Anat-2 monoclonal antibody 6B8 diluted 1:100 in PBS. Coverslips were then washed as described prior to incubation for 1 hour with secondary antibody, fluorescein labelled goat anti-mouse IgG (Pierce, catalog #31569), diluted 1:100 in PBS. Coverslips were washed as described, mounted on slides with Vectashield containing DAPI (Vector Laboratories Inc., catalog #H-1200), and sealed with clear nail polish.

Fluorescence was visualized on a Leica DMLB microscope at 100× magnification under immersion oil (Type DF, Cargille Laboratories Inc., catalog #16424) and imaged using Leica QFISH software version 2.1. The data presented in this FIG. 12 clearly shows surface staining of PA-1 cells, indicating the monoclonal antibody 6B8 recognizes the Anat-2 antigen on the surface of the ovarian tumor cell line.

Immunohistochemical data demonstrating binding of Anat-2 murine monoclonal antibody 6B8 to ovarian tumor samples is depicted in FIG. 13. Human tissue arrays, containing 59 samples each of either diverse normal organs or ovarian carcinoma tissues (Imgenix, cat. nos.

IMH-301 and IMH-347), were stained with either Anti Anat-2 murine monoclonal antibody 6B8 or an isotype-matched negative control (BD Pharmingen, cat. no. 555746). The tissue arrays were first deparaffinized and rehydrated by sequential treatment with heat (5 min at 60° C.), xylene (10 min), ethanol (3 min in 100%, 95% and 70%) and phosphate buffered saline. Each rehydrated slide was incubated 6 min in a hot (80° C.) bath of citrate buffer (Lab Vision, cat. no.

AP-9003-125). After cooling to room temp, the slides were soaked 5 min in 3% hydrogen peroxide in order to reduce potential non-specific effects of endogenous peroxidases. The deparaffinized and rehydrated tissue arrays were incubated 90 min with monoclonal antibodies at a concentration of 0.005 mg/ml. Staining was detected by sequential exposure to a biotin-labeled secondary antibody, avidin-biotin-horseradish peroxidase complex (Vectastain Elite ABC kit cat. no. PK-6102; Vector Laboratories) and diaminobenzidine enzyme substrate (Vector Laboratories, cat. no. SK-4100); all slides were then briefly counterstained with the nuclear dye hematoxylin QS (Vector Laboratories, cat. no. H-3404). Stained tissue arrays were dehydrated in ethanol (70%, 95% and 100%), cleared in xylene and coverslip-mounted with Vectamount (Vector Laboratories, cat. no. H-5000). The slides were viewed with a Nikon Eclipse 600 microscope and digital images acquired by a Spot RT Color digital camera (Diagnostics Instruments Inc.) The data presented in this FIG. 13 shows weak staining of normal ovary (panel B) with Anat-2 monoclonal antibody 6B8, compared to strong staining of ovarian adenocarcinoma (panel D); no staining was detected by the negative control antibody (panels A and C). This data confirms that the Anat-2 antigen is expressed at higher levels in ovarian tumors as compared to normal ovary.

### Example 6

Genbank Accession # AA767317 Example 7 Additional Sequences The following are additional sequences that were identified to be overexpressed in ovarian tumors identified using the GeneLogic Gene Express database. The sequences are listed according to their Genebank accession number from NCB1 database.

Genbank Accession # AA767317 Genbank Accession # AI143233, protein name KIAA0090 Translated protein product from above nucleotide sequence: Genbank Accession # NM-016425, protein name Transmembrane protease, serine 4 Translated protein product from above nucleotide sequence-TMPRSS4 Example 8 Sarcospan Additionally using the GeneLogic database search, we identified a putative ovarian cancer specific splice variant of sarcospan, a known cell surface protein. The nucleotide sequence of this exon (splice variant) and the following a sarcospan gene are respectively contained in SEQ ID NO: 40 and 41 below. As this exon corresponds to a cell surface protein, it is anticipated that antibodies may be produced against this protein and used in the design of prostate cancer therapeutics.

Gene Logic Candidate AW044646 Novel Ovarian Cancer Specific Splice Variant of Sarcospan &gt;gi#5905175#gb#AW044646.1#AW044646 wy78e06.x1 Soares_NSF_F8_{—}9W_OT_PA_P_S1 Homo sapiens cDNA clone IMAGE: 2554690 3′, mRNA sequence &gt;gi#16933560#ref#NM_{—}005086. 3# Homo sapiens sarcospan (Kras oncogene-associated gene) (SSPN), mRNA Example 9 EDG7 Using the same methods, another gene, EDG7, a G protein-coupled receptor, was identified as being overexpressed in ovarian tumors using the Gene Logic Gene Express Oncology Datasuite. The Genbank accession number for this gene is Nom 012152. The nucleotide and protein sequences of EDG7 are set forth below: NM-012152: Homo sapiens endothelial differentiation, lysophosphatidic acid G-protein-coupled receptor, 7 (EDG7), nucleotide sequence NP-036284: Homo sapiens endothelial differentiation, lysophosphatidic acid G-protein-coupled receptor, 7 (EDG7), protein sequence FIG. 3 contains an ‘electronic Northern’ depicting the gene expression profile of this gene as determined using the Gene Logic datasuite. The values along the y-axis represent expression intensities in Gene Logic units. Each blue circle on the figure represents an individual patient sample. The bar graph on the left of the figure depicts the percentage of each tissue type found to express the gene fragment. The total number of samples for each tissue type is as follows: malignant ovary, tumor % above 50, 37; all malignant ovary, 53; normal breast, 30; normal colon, 30; normal esophagus, 17, normal kidney, 27; normal liver, 19; normal lung, 34; normal lymph node 9; normal ovary, 22; normal pancreas, 18; normal rectum, 22; normal spleen, 9; normal stomach, 21.

The expression of EDG7 in normal and malignant human tissues was further investigated by PCR experiments using commercially available human cDNA panels and cDNA samples prepared in-house from human tissues and cell lines. The results of these experiments are presented below in FIGS. 3-5. The following PCR primers were synthesized and used in all experiments.

5′GCTGGAATTGCCTATGTATTCCTGATG 3′ (SEQ ID NO: 47) 5′GCAGCAGGAACCACCTTTTCACAT 3′ (SEQ ID NO: 48) These primers amplify a PCR product of 607 bp from any cDNA template containing the EDG7gene. Expression of Glyceraldehyde 3-phosphate dehydrogenase (GAPDH) is measured in all experiments as a control for cDNA integrity. GAPDH is a housekeeping gene expressed abundantly in all human tissues. Primers used for amplification of the GAPDH gene are: 5′ACCACAGTCCATGCCATCAC 3′ (SEQ ID NO: 49) 5′TCCACCACCCTGTTGCTGTA 3′ (SEQ ID NO: 50) These primers amplify a 482 bp product from any cDNA template encoding the GAPDH gene. For these experiments, an artificial PCR template was generated for use as a positive control for the EDG7 primers. This template was constructed due to the lack of a commercially available plasmid template containing a part of the EDG7 gene. EDG7 primers were synthesized as the 5′ part of the GAPDH primers, to produce the following primer pair: 5′GCTGGAATTGCCTATGTATTCCTGATGACCACAGTCCATGCCATCAC 3′ (SEQ ID NO: 51) 5′GCAGCAGGAACCACCTTTTCACATTCCACCACCCTGTTGCTTA 3′ (SEQ ID NO: 52) This primer pair was used to amplify a PCR product comprising GAPDH sequence flanked by part of the EDG7 sequence using ovarian tumor cell line PA-1 as a template. The PCR product was purified and subsequently used as a positive control with the EDG7 primers described above. EDG7 primers amplify a PCR product of 533 bp from this template. The negative control for all PCR reactions was water (no template.) FIG. 4 shows the expression of EDG7 in normal tissues, as determined using human multiple tissue cDNA panels (MTC panels 1 & 2, BD Biosciences, catalog #s K1420-1 and K1421-1) Upper panel; EDG7 expression, lowerpanel; GAPDH expression. The cDNA samples present in each lane are as follows: 1 heart, 2 brain, 3 placenta, 4 lung, 5 liver, 6 skeletal muscle, 7 kidney, 8 pancreas, 9 spleen, 10 thymus, 11 prostate, 12 testis, 13 ovary, 14 small intestine, 15 colon, 16 peripheral blood leukocyte, 17 negative control, 18 positive control. The arrowhead on the right of the figure denotes the anticipated size of the EDG7 PCR product. The data contained in this figure indicates that EDG7 is expressed weakly in prostate, but is absent from all other normal tissues.

As evidence in the literature suggests that EDG7 is expressed in heart tissue, we investigated this further using a human cardiovascular multiple tissue cDNA panel (BD Biosciences, catalog # K1427-1.) The results of this experiment are presented in FIG. 5.

FIG. 5 shows EDG7 expression in cardiovascular tissue. Upper panel, EDG7 expression; lower panel, GAPDH expression. cDNA samples: 1 adult heart, 2 fetal heart, 3 aorta, 4 apex of the heart, 5 left atrium, 6 right atrium, 7 dextra auricle, 8 sinistra auricle, 9 left ventricle, 10 right ventricle, 11 intraventricular septum, 12 atrioventricular node, 13 negative control, 14 positive control. The arrowhead on the right of the figure denotes the anticipated size of the EDG7 PCR product.

The data presented in this figures indicates that EDG7 is not expressed in any heart tissue, consistent with the data from the MTC panel in FIG. 4.

FIG. 6 shows EDG7 expression in a panel of human ovarian tumor samples and 2 ovarian tumor cell lines. The ovarian tumor samples were obtained from the Cooperative Human Tissue Network (CHTN); the cell lines Ovcar-3 and PA1 were obtained from the American Type Culture Collection (ATCC, Rockville Md.) RNA was isolated from each sample and cell line using Qiagen's RNeasy kit (catalog # 75162). cDNA was prepared from total RNA using SuperScript First Strand Synthesis System for RT-PCR (Invitrogen, catalog &num; 11904-018.) The upper panel shows EDG7 expression, the lower panel shows GAPDH expression. The numbers above each lane correspond to ovarian tumor samples as follows: 1: moderately differentiated cystadenocarcinoma, 2: poorly differentiated papillary serous adenocarcinoma, 3: poorly differentiated papillary serous adenocarcinoma, 4: poorly differentiated endometriod adenocarcinoma, 5: papillary serous adenocarcinoma, 6 endometriod adenocarcinoma, 7: Ovcar-3 cell line, 8: PA-1 cell line, 9: poorly differentiated adenocarcinoma, 10: poorly differentiated papillary serous adenocarcinoma, 11: negative control, 12: positive control. The arrowhead on the right of the figure denotes the anticipated size of the EDG7 PCR product.

The data presented in FIG. 6 indicates that EDG7 is expressed in 5 of 8 tumor samples and both of the ovarian tumor cell lines analyzed. Taken together, the data presented here indicates that EDG7 is highly specific for ovarian tumors, and therefore represents an ideal target for ovarian cancer therapy.

While the invention has been described with respect to certain specific embodiments, it will be appreciated that many modifications and changes thereof may be made by those skilled in the art without departing from the spirit of the invention. It is intended, therefore, by the appended claims to cover all modifications and changes that fall within the true spirit and scope of the invention.

### Example 10

Vaccine Development A significant challenge in vaccine development is selection of antigens capable of inducing a robust CTL response. The MERET gene encodes MHC class I binding peptides, which are effective for inducing a CTL response (Tables 7-11). The nanomer peptides were identified using a combination of approaches essentially as described by Rammensee et al. (1995) Immunogenectis 41: 178; by Parker et al. (1994) J Immunol 152: 163; and by www.Expasy.ch/tools/). Results from peptide analysis programs were expressed as relative scores: score “A” was determined using Parker's method (JImmunol 152:163) and score “B” was determined using Rammensee's method (Immunogenectis 41: 178). The start position refers to the residue of SEQ ID NO: 22 at which the first amino acid of the identified subsequence is found.

The MHC class I binding epitopes are highly conserved. See FIG. 14, which shows an aligment of MERET protein in human and mouse.

Table 7. HLA-A0201 Binding MERET Peptides Peptide St a-(Subsequence Residue Listing Score A Score B 208 YLVEDVLLL 29 I<L C rwisLt 0<1<1z ˜9 1 I 3 25 LL WRKQLFC 385 15 4 141 YLYTANVTL 314 27 5 18 NLLHGLNLL 181 29 sI 566 AVLDDSIYL 155 19 tl 7 515 VMNDRLYA ! 120 25 8 368 VEVENFLFV 98 14 9 60 SLFSSHPPL 79 24 10 30 QLFCDVTLT 63 19 Table 8. HLA-24 Binding MERET Peptides Peptide pos tion Subsequence Residue Listing Score A Score B×1 1 207 KYLVEDVLL 600 25 YAIGGNHL 240 21 217 NFEEMRALL 360 20 217 NFEEMRALL I 360 20 238 LFQMSVLWL 30 17 5 530 GFSHLDVML 24 16_. ______ 6 43 QFHCHKAVL 20 16 I<L M IT L<LmILL 7 285 RTDPVCQKL 17 14 8 396 RYDPRFNSW 17 13 9 322 KMLLLVGGL 14 14 10 366 CWEVENFL 12 13 Table 9. HLA-A3 Binding MERET Peptides Start Subsequence Residue Listing Score A Score B Positon 1 537 MLVECYDPK 45 21 292 KLLLDAMNY 26 XLtIt<1 3 194 ALHGLEETK 30 29 163 ILHIPQVTK 30 31 5 214 LLLNFEEMR 18 18 11 6 445 NLETNEWRY 12 16 1<1 254 XK LL MQYAPDLMK F 9 20 YLVEDVLLL 47 9 341 LVQYYDDEK 6 10 10 297 AMNYHLMPF 6 8 Table 10. HLA-A1 Binding MERET Peptides HI Position SL HL_Start Peptide-.... Subsequence Residue Listing Score A Score B...... _______. _{— — — —} 1 367 WEVENFLF 45 14 NLETNEWRY 26 3 285 RTDPVCQKL 12 24 v 4 481 NGEYVPWLY 11 29 .. _{— —}VILPSCVPY 10 6 245 WLEHDRETR 9 12 SL KLLC 1 606 VAEPLAGPA 15 8 233 ESELALFQM 6 14 9 369 EVENFLFVL 4 17 10 440 SVECYNLET 4 20 XL<S L Table 11. HLA-B7 Binding MERET Peptides Peptide St Subsequence Residue Listing Score A Score B ..... ______ _{—}1 228 LPPPVESEL 80 21 mL 612]<G˜612 ACVTVIL 80 24 3 12 DPSHSDNLL 80 22 _AVLDDSIYL 60 10 5 566 AVLDDStYL 60 10 6 118 SPRA) NNLV 40 10 7 220 EMRALLDSL 40 12 IElLs 1 8 430 GGRNETGYL 40 13 ... _. _{— — —}........... _.______.._{—}.. 9 134 GLRLVLEYL 40 12 10 316 ˆ RIRSNKKML ˆ40T 13 C3<o M Protein, peptide, and nucleic acid vaccines can be prepared using the MERET nucleotide and amino acid sequences disclosed herein. For example, vaccines can be produced recombinantly, optionally using bacterial (e.g., Listeria, Salmonella) or viral (e.g., Vaccinia, Adeno) expression systems.

What is claimed is:

1. An isolated nucleic acid encoding a cancer cell antigen selected from the group consisting of: (a) the nucleotide sequence of any one of SEQ ID NOs: 1, 2, 6, 9, 11, 14, 16, 20, 21, 23, 28, 37, 38, 39, 40, 41, 42, 43, and 44; (b) a nucleotide sequence encoding SEQ ID NO: 22 or 32; and (c) a nucleotide sequence complementary to (a) or (b).

2. The isolated nucleic acid of claim 1, wherein the cancer cell antigen comprises one or more MHC class I binding epitopes.

3. The isolated nucleic acid of claim 1, wherein the cancer cell antigen has a capability to elicit cytotoxic T cell lysis.

4. An isolated nucleic acid comprising a nucleic acid sequence that is at least 70% identical to the sequence of the nucleic acid of claim 1, and which encodes a cancer cell antigen comprising one or more MHC class I binding epitopes.

5. The isolated nucleic acid of claim 4, wherein the nucleic acid sequence is at least 90% identical to the sequence of the nucleic acid of claim 1.

6. The isolated nucleic acid of claim 4, wherein the cancer cell antigen has a capability to elicit cytotoxic T cell lysis.

7. An isolated nucleic acid encoding a cancer antigen comprising one or more MHC class I binding epitopes, which nucleic acid hybridizes to the complement of the nucleic acid of claim 1 under the following stringent conditions: a final wash in 0.1×SSC at 65°.

8. The isolated nucleic acid of claim 7, wherein the cancer cell antigen has a capability to elicit cytotoxic T cell lysis.

9. A diagnostic reagent for detection of cancer comprising a nucleic acid according to claim 1 and a detectable label.

10. A diagnostic reagent comprising primers that result in the specific amplification of the nucleic acid of claim 1.

11. A method for detecting cancer comprising obtaining a human cell sample and detecting a nucleic acid of claim 1 in the cell sample.

12. The method of claim 11, wherein the method comprises detecting specific hybridization to a nucleic acid of claim 1.

13. The method of claim 11, wherein the method comprises amplifying a nucleic acid of claim 1.

14. The method of claim 11, wherein the method comprises detecting a cancer antigen encoded by a nucleic acid of claim 1.

15. The method of claim 14, wherein the detecting comprises binding of an antibody to the cancer antigen.

16. The method of claim 15, further comprising an ELISA or competitive binding assay.

17. A therapeutic reagent comprising a nucleic acid that hybridizes with a nucleic acid of claim 1 and an effector moiety.

18. The therapeutic reagent of claim 17, wherein the effector moiety is a radionuclide, an enzyme, a cytotoxin, a growth factor, or a drug.

19. A method for treating cancer, which comprises administering to a subject in need thereof a therapeutically effective amount of a ribozyme or antisense oligonucleotide that inhibits the expression of a nucleic acid of claim 1.

20. A method for treating cancer, which comprises administering to a subject in need thereof a therapeutic reagent of claim 17.

21. A cancer cell antigen selected from the group consisting of: (a) an antigen encoded by a nucleic acid sequence of claim 1; and (b) fragments or variants of (a) that bind to antibodies that specifically bind the antigen of (a).

22. The antigen of claim 21, wherein the antigen comprises one or more MHC class I binding epitopes.

23. The antigen of claim 22, wherein the one or more MHC class I binding epitopes are selected from the group consisting of an HLA-A0201 binding epitope, an HLA-24 binding epitope, an HLA-A3 binding epitope, an HLA-A1 binding epitope, an HLA-B7 binding epitope, and combinations thereof.

24. The antigen of claim 21, wherein the antigen comprises an amino acid sequence of SEQ ID NO: 22, or MHC class I binding fragment thereof.

25. The antigen of claim 21, wherein the antigen comprises an amino acid sequence of SEQ ID NO: 32, or MHC class I binding fragment thereof.

26. A vaccine comprising an antigen of claim 21 and an adjuvant.

27. The vaccine of claim 26, wherein the antigen comprises one or more MHC class I binding epitopes.

28. The vaccine of claim 27, wherein the one or more MHC-binding epitopes are selected from the group consisting of an HLA-A0201 binding epitope, an HLA-24 binding epitope, an HLA-A3 binding epitope, an HLA-A1 binding epitope, an HLA-B7 binding epitope, and combinations thereof.

29. The vaccine of claim 28, wherein the antigen comprises SEQ ID NO: 22, or MHC class I binding fragment thereof.

30. The vaccine of claim 26, further comprising a capability to elicit a humoral or cytotoxic T lymphocyte response to the antigen.

31. A method for treating cancer, which comprises administering to a subject in need thereof a vaccine comprising a therapeutically effective amount of a vaccine of claim 26.

32. The method of claim 29, wherein the vaccine is administered in combination with a chemotherapeutic agent.

33. A monoclonal antibody or antigen binding fragment thereof, which specifically binds to the antigen of claim 21.

34. The monoclonal antibody of claim 33 which is a chimeric, human, or humanized antibody.

35. A diagnostic reagent comprising an antibody or antigen binding fragment of claim 33 and a detectable label.

36. A therapeutic reagent comprising an antibody or antigen binding fragment of claim 33 and an effector moiety bound.

37. The therapeutic reagent of claim 36, wherein the effector moiety is a radionuclide, an enzyme, a cytotoxin, a growth factor, or a drug.

38. A method for treating cancer, which comprises administering to a subject in need thereof a therapeutically effective amount of an antibody or antigen binding fragment of claim 33.

39. The method of claim 38, wherein the antibody is administered in combination with a chemotherapeutic agent.

40. A method for treating cancer, which comprises administering to a subject in need thereof a therapeutically effective amount of a reagent of claim 36.

41. The method of claim 40, wherein the therapeutic reagent is administered in combination with a chemotherapeutic agent.

42. A monoclonal antibody or antigen binding fragment thereof that specifically binds Anat-2 antigen.

43. The monoclonal antibody of claim 42 which is a chimeric, human, or humanized antibody.

44. A diagnostic reagent comprising an antibody or antigen binding fragment of claim 42 and a detectable label.

45. A therapeutic reagent comprising the monoclonal antibody or antigen binding fragment of claim 42 and an effector moiety.

46. The therapeutic reagent of claim 45, wherein the effector moiety is a radionuclide, an enzyme, a cytotoxin, a growth factor, or a drug.

47. The therapeutic reagent of claim 46, wherein the radionuclide is 90Y or 131I.

48. The monoclonal antibody or antigen binding fragment of claim 42, which does not specifically bind to Anat-1, Anat-3 or Anat-4.

49. A method of treating cancer comprising administering to a subject in need thereof a therapeutically effective amount of the antibody or antigen binding fragment of claim 42.

50. The method of claim 49, wherein the antibody is administered in combination with a chemotherapeutic agent.

51. A method of treating cancer comprising administering to a subject in need thereof a therapeutically effective amount of the therapeutic reagent of claim 45.

52. The method of claim 51, wherein the antibody is administered in combination with a chemotherapeutic agent.
